(19) United States
(12) Patent Application Publication
(10) Pub. No.: US 2008/0166734 A1
(43) Pub. Date: Jul. 10, 2008

(54) Genes and methods of using the same for diagnosis and for targeting the therapy of cardiovascular disease

(76) Inventor: David Xing-Fei Deng, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/315,754

(22) Filed: Dec. 21, 2005

(51) Int. Cl. G01N 33/53

(52) U.S. Cl. 435/7.1

(57) ABSTRACT

The invention is a method of classifying, diagnosing, prognosticating, and predicting cardiovascular disease conditions or other biological states using genes identified using microarray technology.

### REFERENCE TO A COMPUTER PROGRAM LISTING APPENDIX

Two identical Compact Disc-Recordables (CD-Rs) labeled “Copy 1” and “Copy 2” are provided at the Appendix of this patent document. Each CD-R is formatted in IBM-PC format and is compatible with the MS-Windows operating system. Each CD-R includes the following files with the noted creation dates and sizes: Table 1 (dated Dec. 13, 2005; size: 62 kilobytes); Table 2 (dated Dec. 13, 2005; size: 129 kilobytes); Table 3 (dated Dec. 13, 2005; size: 1799 kilobytes); Table 4 (dated Dec. 13, 2005; size: 667 kilobytes); Table 5 (dated Dec. 13, 2005; size: 1316 kilobytes); Table 6 (dated Dec. 13, 2005; size: 991 kilobytes); Table 7 (dated Dec. 13, 2005; size: 24 kilobytes); Table 8 (dated Dec. 13, 2005; size: 33 kilobytes); Table 9 (dated Dec. 13, 2005; size: 34 kilobytes); Table 10 (dated Dec. 13, 2005; size: 233 kilobytes); Table 11 (dated Dec. 13, 2005; size: 96 kilobytes); Table 12 (dated Dec. 13, 2005; size: 68 kilobytes); Table 13 (dated Dec. 13, 2005; size: 23 kilobytes); Table 14 (dated Dec. 13, 2005; size: 35 kilobytes); Table 15 (dated Dec. 13, 2005; size: 40 kilobytes); Table 16 (dated Dec. 13, 2005; size: 52 kilobytes); Table 17 (dated Dec. 13, 2005; size: 2360 kilobytes); Table 18 (dated Dec. 13, 2005; size: 42 kilobytes); Table 19 (dated Dec. 13, 2005; size: 21 kilobytes); Table 20 (dated Dec. 13, 2005; size: 1792 kilobytes); Table 21 (dated Dec. 13, 2005; size: 42 kilobytes); Table 22 (dated Dec. 13, 2005; size: 21 kilobytes); Table 23 (dated Dec. 13, 2005; size: 530 kilobytes); Table 24 (dated Dec. 13, 2005; size: 1373 kilobytes); Table 25 (dated Dec. 13, 2005; size: 496 kilobytes); Table 26 (dated Dec. 13, 2005; size: 236 kilobytes); Table 27 (dated Dec. 13, 2005; size: 2618 kilobytes); Table 28 (dated Dec. 13, 2005; size: 2442 kilobytes); Table 29 (dated Dec. 13, 2005; size: 2066 kilobytes); Table 30 (dated Dec. 13, 2005; size: 1693 kilobytes); Table 31 (dated Dec. 20, 2005; size: 1009 kilobytes); Table 32 (dated Dec. 21, 2005; size: 223 kilobytes); Table 33 (dated Dec. 20, 2005; size: 42 kilobytes); and Table 34 (dated Dec. 20, 2005; size: 11,206 kilobytes).

The content of these files are incorporated by reference herein. The files on each CD-R are accessible using a text-based editor.

### BACKGROUND OF THE INVENTION

Atherosclerosis is a chronic inflammatory disease with lipid deposition and accumulation in vascular walls over many years. Although systemic risk factors such as hyperlipidemia, diabetes mellitus, smoking and hypertension contribute to its development, atherosclerosis preferentially affects certain vascular beds. In the normal anatomical locations, atherosclerosis predominantly affects arteries, and rarely veins. Differences in hemodynamic environments between veins and arteries may play an important role in the development of atherosclerosis but cannot fully explain the differences in predisposition to atherosclerosis between different vascular beds. Even within the same arterial system, atherosclerosis tends to occur focally in certain predisposed regions. For example, the proximal left anterior descending coronary artery in the coronary circulation, the proximal portions of the renal arteries, and the carotid bifurcation in the extracranial circulation exhibit a particular predilection for atherosclerosis. Atherosclerosis also occurs in the aorta, the largest vessel in the body. Therefore, interplay between circulating factors and the properties of the local vascular wall may be critical for the initiation and development of atherosclerosis.

Vascular endothelial cells maintain the interface between the systemic circulation and soft tissues and mediate many critical processes such as inflammation, coagulation and homeostasis. Vascular endothelium is also involved in a diverse array of pathological conditions including atherosclerosis and restenosis.

Smooth muscle cells (SMCs) consist of heterogeneous subtypes among various vascular beds and at different vascular developmental stages (Manabe et al., 2001, J Biol Chem. 276: 39076-3987; Fujita et al., 1993, Exp Mol Pathol. 58: 25-39; Yang et al., 1998, Circulation 97: 181-187). SMCs from veins and arteries have different embryonic origins and are exposed to different hemodynamic environments. However, primary SMC cultures isolated from veins and arteries share many common features including similarities in morphology and responses to mitogens and chemoattractants (Yang et al., 1998; Liu et al., 2004, J Surg Res. 120: 256-265). Atherosclerosis occurs in arteries but rarely in veins under normal anatomical conditions, despite the exposure of all vessels to systemic risk factors such as smoking, hyperlipidemia and hyperglycemia. However, after implantation during arterial bypass surgery saphenous veins become prone to accelerated atherosclerosis (Motwani et al., 1998, Circulation 97: 916-931).

SMC proliferation and migration induced by cytokines, growth factors like platelet-derived growth factor (PDGF), modified lipoproteins like oxidized LDL (OxLDL), and other agents from both vascular cells and infiltrating immune cells, play important roles in many disease processes including atherosclerosis, failure of vascular bypass grafts and post-angioplasty restenosis (Ross R. 1999, N Engl J Med. 340: 115-126; Libby et al., 2002, Nature 420: 868-874). However, the molecular features that determine the different SMC subtypes and unique functional responses are still poorly elucidated.

Gene expression profiling by high-density microarrays is a powerful tool for exploring complex interactive networks of genes and signaling pathways. However, identifying the appropriate biological context for large-scale changes in gene expression may be difficult, and the possible significance of the identification of a group of related genes as differentially regulated has not been adequately addressed with rigorous statistical methodology.

Thus, there remains a need to identify diagnostic markers and therapeutic targets for cardiac disorders, such as atherosclerosis.

### SUMMARY OF THE INVENTION

The invention is a method of classifying, diagnosing, prognosticating about, and predicting cardiovascular disease conditions or other biological states using genes identified using microarray technology.

One aspect of the invention provides for a method for diagnosing a patient or subject having or developing cardiovascular disease comprising detecting a change in expression of at least one of the identified genes that are differentially expressed in coronary artery endothelial cells and /or smooth muscle cells. In some embodiments, the endothelial cells or smooth muscle cells have been exposed to an atherogenic agent. Atherogenic agents can include oxLDL, IL1β, TNFα, and PDGF. The genes showing differential expression in endothelial cells and smooth muscle cells in the presence of an atherogenic agent are compared to either endothelial or smooth muscle cells in the absence of the agent and/or to saphenous vein cells similarly treated.

A method for diagnosis of cardiovascular disease or susceptiblity to cardiovascular disease comprises detecting the expression of one or more genes, for example, as listed in Tables 3-4, 23-24, and 32-33 in a biological sample, and determining whether the level of expression of the gene matches the level of expression of the gene in a endothelial cell and/or smooth muscle cell treated with an atherogenic agent. In some embodiments, the expression of at least two, three, four, five, six, seven, eight, nine, ten or all of the genes shown in Table 3-4, Table 23-24, or Tables 32-33 are detected. In some embodiments, the genes are upregulated or downregulated at least 1.5 fold as compared to untreated cells under the same conditions. In other embodiments, the genes are upregulated or downregulated at least about 3 fold and more preferably 10 fold. In other embodiments, the genes that are identified for diagnostic or therapeutic purposes are those that are upregulated or downregulated compared to control with at least a P value <0.0001, preferably a P value up to 10⁻⁸ and are optionally, upregulated or downregulated at least 3 fold as compared to control.

In some embodiments, the biological sample is serum or blood. In other embodiments, the biological sample is coronary artery smooth muscle cells or endothelial cells.

In some embodiments, the expression of the genes is detected by detecting the polypeptide. Sequence of polypeptides encoded by the genes can be determined using publicly accessible databases and the gene names. In addition, Table 31 provides for the accession numbers of the genes as well as the Agilent probe identification number which will allow for identification of the sequences of the polypeptides as well as probes that can detect the sequences. In some embodiments, the polypeptide is a secreted polypeptide and may be measured in blood or serum. In some embodiments, the expression of the polypeptide is increased at least 1.5 fold, more preferably about 5 fold and more preferably about 10 fold or more above untreated cells under the same conditions. In other embodiments, the expression of the polypeptide is decreased about 1.5 fold, more preferably 3 fold and more preferably, 10 fold.

In some embodiments, expression of the polypeptide is detected using antibodies that specifically bind to the polypeptide. In some embodiments, the antibody is detectably labelled. The labels can include, for example, green fluorescent protein, enzymes, biotin, fluorescent moieties, and radioactive moieties. In other embodiments, an activity assay might be utilized to detect the polypeptide. Such activity assays depend on the nature of the polypeptide and many are known in the art and may be described below.

In other embodiments, expression of the gene may be detected with a primer or probe that specifically recognizes the gene. Such probes have been described and identified on the array such as those of Agilent and the Agilent probe identification numbers are provided in Table 34. These numbers can be utilized to determine the sequence of the probes for detecting any of the genes described herein. Alternatively, other databases might provide for cloned sequences that can detect a particular gene. Primers can be designed to amplify a particular sequence in accord with methods known in the art and can be utilized in PCR methods such as real time PCR.

Several types of genes have been identified as being associated with coronary artery smooth muscle cells and endothelial cells that have been treated with an atherogenic agent. The expression profile of one or more of these genes can be utilized to diagnose cardiovascular disease or susceptibility to cardiovascular disease.

In a specific embodiment, a method for diagnosing cardiovascular disease or susceptibility to cardiovascular disease comprises detecting a CXCL12 polypeptide or an increase in expression of a polynucleotide encoding a CXCL12 polypeptide in a biological sample, wherein an increase in CXCL12 polypeptide or an increase in expression of a polynucleotide encoding a CXCL12 polypeptide as compared to control sample is indicative of cardiovascular disease. The CXCL12 polypeptide may be detected using an antibody or an activity assay. An activity assay can comprise measuring chemotactic activity for lymphocytes of CXCL12 or the cleavage of CXCL12 by matrix metalloproteinase-2. An increase in expression of a polynucleotide encoding a CXCL12 polypeptide can be detected using a probe that specifically binds to CXCL12 or a primer and PCR. The method is useful in the diagnosis of cardiovascular condition selected from the group consisting of atherosclerosis, stroke, ischemic heart disease, hypertension, cardiac hypertrophy, post-angioplasty restenosis, angina, and coronary heart disease. In some embodiments, the biological sample comprises aortic smooth muscle cells or aortic endothelial cells. In other embodiments, the biological sample comprises blood or serum.

In some embodiments, the method further comprises detecting a CXCL11 or CXCL10 polypeptide or both, wherein a decrease in the CXCL11 or CXCL10 polypeptide or both as compared to a control is indicative of cardiovascular disease. The detecting CXCL11 or CXCL10 polypeptide, or both, may be detected with a specific antibody that binds to CXCL11 or CXCL10, respectively. In some embodiments, a decrease in the expression of a polynucleotide encoding a CXCL11 or CXCL10 polypeptide or both as compared to a control is detected. In some embodiments, a decrease in expression is detected with a probe or primer.

One aspect of the invention includes a method of targeting a product of at least one of the genes in tables 3, 23, 32, or 33 that includes identifying a therapeutic agent having a therapeutic effect on said gene product. Another embodiment includes a method of targeting a product of at least one of the genes in Tables 20, 27, and 29 for identification of an antagonist or agonist that can be utilized to treat cardiovascular disease.

Another aspect of the invention involves a method of screening for an agent that modulates a gene or polynucleotide for treatment for cardiovascular disease. A screening method comprises a method for detecting an agent that can modulate the expression of at least one gene or polynucleotide for which a change in expression is is seen in a cardaic aortic cell treated with an atherogenic agent. In some embodiments, the genes or polynucleotides are selected from the genes of Table 3, Table 23, Table 32, or Table 33. In other embodiments, the genes are selected from those that have at least 3 fold upregulation or down regulation, more preferably have a P value less than 0.0001, more preferably less than 10⁻⁸.

If a gene is upregulated, a method comprises identifying an antagonist of the gene or polynucleotide. In some embodiments, a method for identifying an antagonist comprises contacting one or more or preferably all of the genes or polypeptides encoded by the genes that are upregulated at least 1.5 fold in coronary artery smooth muscle cells or endothelial cells in response to an atherogenic stimuli with a candidate molecule and determining whether the candidate molecule inhibits expression or activity of the gene or polypeptide. The upregulated genes are shown in Tables 3, 5, 20, 23, 27, 29, 32 and 33. In some embodiments, the atherogenic stimulus is selected from the group consisting of oxLDL, TNFα, IL1β and PDGF. In some embodiments, the genes include chemokines, endothelial and smooth muscle markers, cell proliferation and apoptotic genes, Alzheimer's related genes, TNFα stimulated genes, and IL1β stimulated genes as described above.

If a gene is downregulated, a method comprises identifying an agonist of the gene or polynucleotide. In some embodiments, a method for identifying an agonist comprises contacting one or more or preferably all of the genes or polypeptides encoded by the genes that are downregulated at least 1.5 fold in coronary artery smooth muscle cells or endothelial cells in response to an atherogenic stimuli with a candidate molecule and determining whether the candidate molecule increases expression or activity of the gene or polypeptide. The downregulated genes are shown in Tables 3, 5, 20, 23, 27, 29, 32, and 33. In some embodiments, the atherogenic stimuli is selected from the group consisting of oxLDL, TNFα, IL1β and PDGF. In some embodiments, the genes include chemokines, endothelial and smooth muscle markers, cell proliferation and apoptotic genes, Alzheimer's related genes, TNFα stimulated genes, and IL1β stimulated genes as described above.

Exemplary sequences for the genes, polynucleotides, or polypeptides of can be found in Accession Nos. as provided in Tables 3, 5, 20, 23, 27, 29, 31, 32, and 33. Complementary sequences for the genes and polynucleotides can readily be determined by one of skill in the art.

In a specific embodiment, a method for screening for an antagonist of CXCL12 comprises contacting a CXCL12 polypeptide with an agent; and determining whether the agent inhibits binding of CXCL12 to its receptor CXCR4 or inhibits an activity of the CXCL12. In some embodiments, wherein the agent is an antibody or an aptamer. In some embodiments, inhibition of activity is measured by chemoattraction of lymphocytes or cleavage of CXCL12 by matrix metalloproteinase-2. In an alternative embodiment, a method for screening for an antagonist of CXCL12 comprises contacting polynucleotide encoding a CXCL12 polypeptide, with a candidate agent, and determining whether the candidate agent inhibits expression of the polypeptide. In som embodiments, the agent is selected from the group consisting of an antisense molecule, siRNA and peptide nucleic acid.

The invention also provides for kits for diagnosis, including one or more agents for detecting the diagnostic markers, as well as, instruction for detecting the diagnositc markers.

### BRIEF DESCRIPTION OF THE FIGURES

FIG. 1. Cell Proliferation and Migration in CASMC and SVSMC. Bar graph represents the fold change of treated vs untreated cells for cell proliferation (A) and migration (B). Cultured cells were treated with OxLDL, oxidized low density lipoprotein; TNFα, Tumor Necrosis Factor α, TNFβ, Tumor Necrosis Factor β; PDGF, Platelet-derived Growth Factor; and FBS, fetal bovine serum. Results were from three separate cell cultures with 6 replicates for each treatment condition.

FIG. 2. Proliferative and Migratory Responses to OxLDL and PDGF in CASMC and SVSMC. Bar graph represents the fold change of treated vs untreated cells for cell proliferation (A) and migration (B). Results were from three separate cell cultures with 6 replicates for each treatment condition.

FIG. 3. Inhibition of SVSMC Proliferation by an Antibody against IGF1 Receptor. Percentage of inhibition of SVSMC proliferation by 25 μg/ml of anti-IGF1R antibody, which was pre-incubated in serum-free medium for 24 h before adding OxLDL or PDGF, as indicated. Results shown are one representative cell culture of two separate cultures assayed with 6 replicates for each treatment condition.

FIG. 4. OxLDL induces cell proliferation in CAEC but not in SVEC. Cultured cells were stimulated with OxLDL, and proliferation quantified by BrdU labeling. CTL, control; OxLDL, oxidized low density lipoprotein; FBS, fetal bovine serum.

FIG. 5. Confirmation of microarray results with Taqman® Real-Time RT-PCR. Bar graph represents the relative ratios of CASMC vs SVSMC for the selected genes. Real Time RT-PCR was used to quantify the selected gene expression levels for the same RNA samples hybridized on arrays.

### BRIEF DESCRIPTION OF THE TABLES

Table 1. List of significant GO terms. All terms with p value ≦0.005 were listed. P value and Corrected p-Value as well as gene count were given to each term.

Table 2. List of significant PA terms. All terms with p value ≦0.005 were listed. Z score, P value, and Bonferroni Corrected p-Value were given to each term. Negative z score indicates that the term is associated with down-regulated genes, or visa versa.

Table 3. OxLDL-induced differentially expressed genes in CASMC. Genes with fold change ≧1.5 and p value <0.001 were selected using Resolver® System (available from Rosetta, Inc., Seattle, Wash.). Negative fold change indicates down-regulated genes after the treatment, or vice versa.

Table 4. OxLDL-induced differentially expressed genes in SVSMC. Genes with fold change ≧1.5 and p value <0.001 were selected using Resolver® System. Negative fold change indicates down-regulated genes after the treatment, or vice versa.

Table 5. PDGF-induced differentially expressed genes in CASMC. Genes with fold change ≧1.5 and p value <0.001 were selected using Resolver® System. Negative fold change indicates down-regulated genes after the treatment, or vice versa.

Table 6. PDGF-induced differentially expressed genes in SVSMC. Genes with fold change ≧1.5 and p value <0.001 were selected using Resolver® System. Negative fold change indicates down-regulated genes after the treatment, or vice versa.

Table 7. Number of Genes up-regulated or down-regulated by PDGF and OxLDL in CASMC and SVSMC.

Table 8. Over-represented pathways from statistical analysis of pathway (PA) terms. The table shows the significant terms for pathway analysis. Terms associated with upregulated genes over control (R) or terms over-represented in untreated CASMC; Terms associated with downregulated genes over control (G), or terms over-represented in untreated SVSMC. Blank cells indicate a P>0.005. CA, CASMC; SV, SVSMC. Terms with P ≦0.005 are considered significant. Not all significant terms are shown. The full list of significant PA is shown in Table 2.

Table 9. Over-represented pathways from statistical analysis of gene ontology (GO) terms. The table shows the significant terms for GO analysis. Terms associated with upregulated genes over control (R) or terms overrepresented in untreated CASMC; Terms associated with downregulated genes over control (G), or terms over-represented in untreated SVSMC. Blank cells indicate a P>0.005. CA, CASMC; SV, SVSMC. Terms with P ≦0.005 are considered significant. Not all significant terms are shown. The full list of significant GO terms is shown in Table 1.

Table 10. Genes that have consistent changes between CASMC and SVSMC. Genes with fold change ≧1.5 and p value <0.001 were selected using Resolver® System. Negative fold change indicates the gene expression is lower in CASMC than SVSMC, or visa versa.

Table 11. List of genes with opposite changes between CASM and SVSM in response to OxLDL. Negative ratio fold changes represent down-regulated genes in response to OxLDL.

Table 12. List of genes with opposite changes between CASM and SVSM in response to OxLDL. Negative ratio fold changes represent down-regulated genes in response to OxLDL.

Table 13. IGFBP Genes Differentially Expressed in CASMC and SVSMC. Upregulated genes (R) over control, or genes having a higher level in untreated CASM; Downregulated genes (G) over control, or genes having a higher level in untreated SVSM. CA, CASM; SV, SVSM. Only significant genes with a P value ≦0.001 and fold change ≧1.5 are shown. Blank cells indicate that the P value and fold change did not meet this requirement.

Table 14. Chemokine Genes Differentially Expressed in CASMC and SVSMC. Upregulated genes (R) over control, or genes having a higher level in untreated CASM; Downregulated genes (G) over control, or genes having a higher level in untreated SVSM. CA, CASM; SV, SVSM. Only significant genes with a P value ≦0.001 and fold change ≧1.5 are shown. Blank cells indicate that the P value and fold change did not meet this requirement.

Table 15. Cytokine Genes Differentially Expressed in CASMC and SVSMC. Upregulated genes (R) over control, or genes having a higher level in untreated CASM; Downregulated genes (G) over control, or genes having a higher level in untreated SVSM. CA, CASM; SV, SVSM. Only significant genes with a P value ≦0.001 and fold change ≧1.5 are shown. Blank cells indicate that the P value and fold change did not meet this requirement.

Table 16. Extracellular Matrix Genes Differentially Expressed in CASMC and SVSMC. Upregulated genes (R) over control, or genes having a higher level in untreated CASM; Downregulated genes (G) over control, or genes having a higher level in untreated SVSM. CA, CASM; SV, SVSM; Matrix Metalloproteinase, MMP; Tissue Inhibitors of Metalloproteinases, TIMP; Collagens, COL; Integrins, ITG. Only significant genes with a P value ≦0.001 and fold change ≧1.5 are shown. Blank cells indicate that the P value and fold change did not meet this requirement.

Table 17. Differeintially expressed genes between untreated CASMC and untreated SVSMC. Genes with fold change ≧1.5 and p value <0.001 were selected using Resolver® System. Negative fold change indicates the gene expression is lower in CASMC than SVSMC, or visa versa.

Table 18. OxLDL-Induced Expression Changes of Endothelial and Smooth Muscle Genes in CASMC and SVSMC. Negative ratio fold changes represent down-regulated genes after OxLDL.

Table 19. Comparison of results from arrays and QuantiGene® assays.

Table 20. Genes differentially expressed between untreated CAEC and SVEC. Cells were cultured in medium with minimally required growth factors without serum for 36 hrs before harvesting for RNA. Ratio fold changes were computed by Rosetta Resolver® System using an error-weighted approach where expression log ratios with smaller error bars contribute more to the combined result than those with larger error bars. Error weighted one-way ANOVA was used in the comparisons between two different cell types. All genes listed were P<0.005 between two cell types and sorted by fold change. “-” represents fold change higher in SVEC than CAEC or vice versa.

Table 21. Selected genes over-expressed in SVEC over CAEC in basal condition.

Table 22. GO terms associated with differentially expressed genes between untreated SVEC and CAEC. “G” indicates over-represented terms associated with genes over-expressed in SVEC; “R” indicates over-represented terms associated with genes over-expressed in CAEC. All terms shown have a P value <0.005.

Table 23. OxLDL-induced differentially expressed genes in CAEC. Cells were treated with fully OxLDL (40 ug/ml) for 24 hours after cultured in serum-free medium for 12 hrs. All genes listed were P<0.005 between OxLDL-treated and untreated cells and sorted by fold change. “-” represents down-regulated genes after OxLDL.

Table 24. OxLDL-induced differentially expressed genes in SVEC. Cells were treated with fully OxLDL (40 ug/ml) for 24 hours after cultured in serum-free medium for 12 hrs. All genes listed were P<0.005 between OxLDL-treated and untreated cells and sorted by fold change. “-” represents down-regulated genes after OxLDL.

Table 25. Over-represented GO terms for all cell types and treatments. All genes were assigned to corresponding GO terms. The p value for each term was calculated by comparing the number of genes annotated by the GO to the expected number of genes annotated by this term in the whole array using hypergeometric distribution. GO terms with P values <0.005 were considered significant. Bonferroni corrected p values were also computed for multiple testing.

Table 26. Over-represented Pathway terms for all cell types and treatments with P value and gene count. All genes were assigned to corresponding pathway terms. The Z score for each term was calculated by comparing the number of genes annotated by the pathway term to the expected number of genes annotated by this term in the whole array using hypergeometric distribution. Pathway terms with Z scores >3 or P value <0.005 considered significant. Z scores or P value with “-” indicate terms associated with genes down-regulated after the treatment or terms associated with genes higher in SVEC than CAEC in untreated cells.

Table 27. TNFα-induced differentially expressed genes in CAEC. Cells were treated with TNFα (10 ng/ml) for 24 hours after cultured in serum-free medium for 12 hrs. All genes listed were P<0.005 between TNFα-treated and untreated cells and sorted by fold change. “-” represents down-regulated genes after TNFα.

Table 28. TNFα-induced differentially expressed genes in SVEC. Cells were treated with TNFα (10 ng/ml) for 24 hours after cultured in serum-free medium for 12 hrs. All genes listed were P<0.005 between TNFα-treated and untreated cells and sorted by fold change. “-” represents down-regulated genes after TNFα.

Table 29. IL1β-induced differentially expressed genes in CAEC. Cells were treated with fully IL1β (10 ng/ml) for 24 hours after cultured in serum-free medium for 12 hrs. All genes listed were P<0.005 between IL1β-treated and untreated cells and sorted by fold change. “-” represents down-regulated genes after IL1β.

Table 30. IL1β-induced differentially expressed genes in SVEC. Cells were treated with fully IL1β (10 ng/ml) for 24 hours after cultured in serum-free medium for 12 hrs. All genes listed were P<0.005 between IL1β-treated and untreated cells and sorted by fold change. “-” represents down-regulated genes after IL1β.

Table 31. Genes differentially expressed between untreated CAEC and SVEC. Cells were cultured in medium with minimally required growth factors without serum for 36 hrs before harvesting for RNA. Ratio fold changes were computed by Rosetta Resolver® System using an error-weighted approach where expression log ratios with smaller error bars contribute more to the combined result than those with larger error bars. Error weighted one-way ANOVA was used in the comparisons between two different cell types. All genes listed were P<0.005 between two cell types and sorted by fold change. “-” represents fold change higher in SVEC than CAEC or vise versa.

Table 32. OxLDL-induced differentially expressed genes in CASMC. Genes with fold change ≧3.0 and p value <10⁻⁸ were selected using Resolver® System (available from Rosetta, Inc., Seattle, Wash.). Negative fold change indicates down-regulated genes after the treatment, or vice versa.

Table 33. OxLDL-induced differentially expressed genes in SVSMC. Genes with fold change ≧3.0 and p value <10⁻⁸ were selected using Resolver® System. Negative fold change indicates down-regulated genes after the treatment, or vice versa.

Table 34. Probes used in gene expression profiling.

### DETAILED DESCRIPTION OF THE PREFERRED EMBODIMENTS

### Definitions

The term “antibody” is used in the broadest sense and specifically includes monoclonal antibodies (including full length monoclonal antibodies), multispecific antibodies (e.g., bispecific antibodies), and antibody fragments that exhibit a desired biological activity or function.

Antibodies can be chimeric, humanized, or human, for example, and can be antigen-binding fragments of these. Antibodies are generally produced by immunizing an animal with an antigen, and can be produced by recombinant technology, or by synthesis of the amino acid sequence, for example. “Antibody fragments” comprise a portion of a full-length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies such as bispecific antibodies, for example formed from antibody fragments. “Functional fragments” substantially retain binding to an antigen of the full-length antibody, and retain a biological activity.

The term “antisense molecule” or “antisense RNA” refers to a polynucleotide that is a complement to a message (or “sense”) strand of RNA. The antisense molecule can form a duplex with a sense strand of RNA (mRNA). This duplex can block translation of the mRNA into a polypeptide by blocking a ribosome's access to the mRNA or a RNA duplex can be degraded by ribonucleases.

The term “aptamer” refers to functional nucleic acids that have the ability to bind almost any target protein with high affinity and specificity. Because of the small molecule-like binding characteristics of aptamers, they typically show strong inhibition of protein function.

The term “atherosclerosis” refers to a disease of large and medium-sized arteries and is characterized by endothelial dysfunction, vascular inflammation, and the buildup of lipids, cholesterol, calcium, and cellular debris within the intima of the vessel wall. This buildup results in plaque formation, vascular remodeling, acute and chronic luminal obstruction, abnormalities of blood flow, and diminished oxygen supply to target organs.

The term “biological sample” refers to a body sample from any animal, but preferably is from a mammal, more preferably from a human. Such samples include biological fluids such as serum, plasma, vitreous fluid, lymph fluid, synovial fluid, follicular fluid, seminal fluid, amniotic fluid, milk, whole blood, biopsy material, urine, cerebro-spinal fluid, saliva, sputum, tears, perspiration, mucus, and tissue culture medium, as well as tissue extracts such as homogenized tissue, and cellular extracts.

The term “cardiovascular disease” refers to any pathology of the heart and/or blood vessels. Diseases or pathologic conditions of blood vessels include aneurysms, atherosclerosis, arteriolosclerosis, and the like. Vascular pathology can includes changes in hemodynamics, blood flow, etc., and can lead to other cardiovascular disease states including, but not limited to, stroke, coronary heart disease, myocardial infarction, hypertension, congestive heart failure, angina, postangioplasty restenosis, failure of vascular bypass grafts, and the like.

The term “chemokine” refers to a group of structurally related small molecules (approximately 8-14 KD) that activate leukocytes and are induced by proinflammatory stimuli. Chemokines regulate many cellular functions of leukocytes through interactions with specific membrane G protein-coupled receptors. Chemokines also play fundamental roles in the development, homeostasis, and function of the immune system, and they have effects on cells of the central nervous system as well as on endothelial cells involved in angiogenesis. Structurally, chemokines are divided into 2 major subfamilies, CXC (α chemokines) and CC (β chemokines), based on the arrangement of the first 2 of the 4 conserved cysteine residues; the 2 cysteines are separated by a single amino acid in CXC chemokines and are adjacent in CC chemokines. CXC chemokines are further subdivided into ELR and non-ELR types based on the presence or absence of a glu-leu-arg sequence adjacent to the CXC motif. ELR types are chemotactic for neutrophils, while non-ELR types are chemotactic for lymphocytes.

A “control” is an alternative subject or sample used in an analytical procedure for comparison purposes. A control can be “positive” or “negative”. For example, where the purpose of an analytical procedure is to detect a differentially expressed transcript or polypeptide in cells or tissue affected by a disease of concern, it is generally preferable to include a positive control, such as a subject or a sample from a subject exhibiting the desired expression and/or clinical syndrome characteristic of the desired expression, and a negative control, such as a subject or a sample from a subject lacking the desired expression and/or clinical syndrome of that desired expression.

The term “detecting” is used in the broadest sense to include both qualitative and quantitative measurements of a specific molecule, for example, measurements of a specific molecule such as a chemokine.

“Differentially expressed,” as applied to nucleotide sequence or polypeptide sequence in a subject, refers to over-expression or under-expression of that sequence when compared to that detected in a control. Underexpression also encompasses absence of expression of a particular sequence as evidenced by the absence of detectable expression in a test subject when compared to a control.

As used herein, “expression” refers to the process by which a polynucleotide is transcribed into mRNA and/or the process by which the transcribed mRNA (also referred to as “transcript”) is subsequently translated into peptides, polypeptides, or proteins.

The term “matrix metalloproteinases” or “MMPs” refers to zinc-dependent endopeptidases. MMPs are capable of degrading all kinds of extracellular matrix proteins, but also can process a number of bioactive molecules. MMPs are involved in cleavage of cell surface receptors, release of apoptotic ligands, and chemokine in/activation. MMPs are also thought to play roles in cell proliferation, migration, differentiation, angiogenesis, apoptosis, and host defense.

The terms “nucleic acid,” “nucleotide,” “polynucleotide,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Polypeptide” refers to a peptide or protein containing two or more amino acids linked by peptide bonds, and includes peptides, oligimers, proteins, and the like. Polypeptides can contain natural, modified, or synthetic amino acids. Polypeptides can also be modified naturally, such as by post-translational processing, or chemically, such as amidation acylation, cross-linking, and the like.

“Polymerase chain reaction” or “PCR” refers to a procedure or technique in which minute amounts of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51:263 (1987); Erlich, ed., PCR Technology, (Stockton Press, N.Y., 1989).

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

The term “probe” refers to an oligonucleotide that hybridizes to a target sequence. In the TaqMan® or TaqMan®-style assay procedure, the probe hybridizes to a portion of the target situated between the annealing site of the two primers. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides. A probe can further include a detectable label, e.g., a fluorophore (Texas-Red®, Fluorescein isothiocyanate, etc.,). The detectable label can be covalently attached directly to the probe oligonucleotide, e.g., located at the probe's 5′ end or at the probe's 3′ end. A probe including a fluorophore may also further include a quencher, e.g., Black Hole Quencher™, Iowa Black™, etc.

The term “Peptide nucleic acids” or “PNA” refers to DNA mimics with a pseudopeptide backbone. PNA is an extremely good structural mimic of DNA (or RNA), and PNA oligomers are able to form very stable duplex structures with complementary DNA, RNA (or PNA) oligomers, and can also bind to targets in duplex DNA by helix invasion.

The term “short interfering RNA” or “siRNA” refers to a class of RNA molecules, typically 20-25 nucleotide-long, that can interfere with the expression of genes. siRNAs are fragments of the antisense strand of RNA that can bind to the complementary sense strand of mRNA, thereby “silencing” that transcript. Some siRNAs can interfere with transcription by binding to complementary sequences on DNA or binding to the nascent RNA transcript to prevent the completion of transcription. siRNAs are naturally produced as part of the RNA interference (RNAi) pathway by the enzyme Dicer. They can also be exogenously introduced to “silence” or knockout gene expression.

The term “significant” or “significantly”, as used herein, refers to statistical significance. Statistical significance is P<0.05, unless a correction is applied to correct for a confounding influence (e.g., Bonferroni's correction).

### Diagnosis and Therapeutic Targets of Cardiovascular Disease Using Gene Expression Profiling

In an embodiment, diagnosis and identification of therapeutic targets for cardiovascular disease can be made using gene expression data and a method of analyzing the data.

Atherosclerosis occurs predominantly in arteries and only rarely in veins. Gene expression profiles of endothelial cells (ECs) from human saphenous vein (SVEC) and coronary artery (CAEC) can be compared in the presence or absence of stimuli in order to determine any differences in gene expression. In some embodiments, the atherogenic stimuli comprises oxidized low density lipoprotein (OxLDL), interleukin 1β (IL1β), or tissue necrosis factor α (TNFα) or mixtures thereof. Vascular endothelial cells maintain the interface between the systemic circulation and soft tissues and mediate many critical processes such as inflammation, coagulation and homeostasis. Vascular endothelium is also involved in a diverse array of pathological conditions including atherosclerosis and restenosis.

Smooth muscle cells (SMCs) from veins and arteries have different embryonic origins and are exposed to different hemodynamic environments. Gene expression profiles of smooth muscle cells (SMCs) from human saphenous vein (SVSMC) and coronary artery (CASMC) can be compared in the presence or absence of stimuli in order to determine any differences in gene expression. In some embodiments, the atherogenic stimuli comprises oxidized low density lipoprotein (OxLDL), or Platelet Derived Growth Factor (PDFG) or mixtures thereof. SMC proliferation and migration play important roles in many disease processes including atherosclerosis, failure of vascular bypass grafts and post-angioplasty restenosis.

### Gene Expression Profiles

Experimental data utilized in methods of the invention is high dimensional data such as gene expression data. Gene expression data are high dimensional data because each sample has a large number of gene expression levels. Generally speaking, gene expression data generally have thousands of gene expression levels for each sample. Other examples of high dimensional data useful in the invention include but are not limited to protein arrays and protein chips, cell array based expression analysis, analysis of patterns of single nucleotide polymorphisms in disease conditions, and comparative genomic hybridization on metaphase, BAC genomic, cDNA and oligonucleotide arrays.

Preferably, gene expression data are obtained through use of DNA microarray technology. DNA microarrays are preferred as a source of data because they generally offer a more complete picture of the interactions of a large number of genes with a limited number, or even one experiment. In some embodiments, gene expression profiles are compared between coronary artery and saphenous vein cells that have been treated with atherogenic stimuli such as oxidized low density lipoprotein (OxLDL), interleukin 1β (IL1β), tissue necrosis factor α (TNFα) or Platelet Derived Growth Factor (PDFG) or mixtures thereof An example of a general description of how gene expression data can be obtained by using DNA microarray technology is given in the examples below.

DNA microarrays, although a relatively new technology, have already been saddled with a number of different names, biochip, DNA chip, gene chip, genome chip, cDNA microarray, and gene array. The use of any of these terms herein refers generally to DNA microarrays. The underlying principle of DNA microarrays is base pairing or hybridization i.e., A-T and G-C for DNA, and A-U and G-C for RNA.

DNA microarrays provide a medium for matching known and unknown DNA samples based on the base pairings given above. DNA microarrays can either be fabricated by high-speed robotics or can be fabricated in a laboratory setting. They are generally patterned on glass, but can also be fabricated on nylon substrates. Microarrays generally have sample spot sizes of less than 200 μm diameter, and generally contain thousands of DNA spots on one microarray.

DNA microarrays are also commercially available from a number of sources, including but not limited to Affymetrix, Inc. (Santa Clara, Calif.), Agilent Technologies (Palo Alto, Calif.), Invitrogen, and Research Genetics (Huntsville, Ala.). Other polynucleotide sequences useful for detecting expression of a gene can be found in the Unigene database or at The Image Consortium. In an embodiment, the array utilized is Agilent human 1A oligo array representing 16,391 unigene sequences (http://www.chem.agilent.com). The sequences on the array for detecting any of the genes are provided at the Agilent website and are readily available, and are also described in Table 31 by the Agilent probe identifier. The gene sequences detectable by the array are identified by Accession Nos. and gene names.

A biological sample is obtained and the mRNA is isolated and hybridzed to the array and expression levels analyzed using standard methodologies. In some embodiments, the biological sample is selected from the group consisting of aortic endothelial cells, aortic smooth muscle cells, saphenous vein endothelial cells, and saphenous vein smooth muscle cells. In some embodiments, the cells have been treated with an atherogenic agent selected from the group consisting of OxLDL, ILIβ, TNFα and PDGF.

### Data Analysis

Data from the expression arrays scanned at two different PMT settings are combined, and imported into the Resolver System (Rosetta, Inc., Seattle, Wash.). Combined gene expression ratios are computed by using an error-weighted approach where expression log ratios with less error contribute more to the combined result than those with greater error. Error weighted one-way ANOVA is used in the comparisons between two different cell states. Fold changes of gene expression >1.5 fold and ANOVA P values <0.005 are considered significant. Differentially expressed genes are selected on strict criteria of P value <0.005 and fold change >1.5 to reduce false positives. Reproducibility of microarray results as measured by the co-efficient of variation between arrays for signal intensities in the reference RNA channel is measured and preferably, falls within 10.5% for all probes and 6.8% for probes with signal intensities in the top 75%. In some embodiments, expression level of five genes can be selected to compare the results between microarrays and QuantiGene® assays in untreated and treated cells, and consistency of the values can be correlated.

In some embodiments, the data are analyzed for gene ontogeny (GO) and pathway analysis relationships (PA). This analysis is useful to identify biological pathways or categories of molecules that can serve as biomarker or therapeutic targets for cardiovascular disease and provide a basis for prioritizing the potential targets or biomarkers for development.

Each list of differentially expressed genes is analyzed in the context of GO terms in order to identify groups of genes with similar functions or processes. GO annotation for genes represented on Agilent human 1A oligo arrays is obtained using Biomolecule Naming Service (Kincaid et al., BNS: An LDAP-based Biomolecule Naming Service. In: OiBC. Washington DC; 2002). BNS is a high-speed directory service developed at Agilent Laboratories, which can resolve between alias and official gene symbols, as well as between different gene identifier schemes, and links to publicly available databases. Of the total 16,400 genes on the array, 9828 genes annotated with GO terms are analyzed, with 8679 genes of cellular component, 7809 genes of biological process and 7562 genes of molecular function. In a specific embodiment, like treated vs non-treated cells, a list of genes with fold change >1.5 and ANOVA P value <0.005, is analyzed by GO and pathway analysis using similar methods as described by Doniger et al. (2003, Genome Biol. 4:R7).

In one embodiment, Gene g is linked to a GO term t, if the GO annotation for gene g contained term t or a child of term t. For each GO term t, whether a term t is over- or under-represented in a given list of genes is tested against the null hypothesis that the distribution of terms is random. This term to gene list association is measured using a hypergeometric distribution as the probability of observing k or more genes annotated by term t in the set of n genes, when there are K genes annotated by term t in the whole set of N genes. Namely, for each term t and list of genes 1, the P value is calculated as follows:

$$p ( t , 1 ) = 1 - \frac{{\sum }_{y = 0}^{k} ( \begin{matrix}n \\ y\end{matrix} ) ( \begin{matrix}N - n \\ K - y\end{matrix} )}{( \begin{matrix}N \\ K\end{matrix} )} .$$

where:
N=the total number of unique genes represented in the microarray, n=the total number of unique genes represented in the set, K=the total number of entries in N mapping to a specific pathway, p, and k=the number of entries in n mapping to a specific pathway. P values are Bonferroni corrected for multiple testing. Similar approaches for Gene Ontology analysis have been reported (Al-Shahrour et al., 2004, Bioinformatics. 20: 578-580; Zeeberg et al., 2003, Genome Biol. 4:R28). Terms with p<0.005 are considered significant.

BNS can also be used to link genes represented on the microarray to the list of 360 pathway terms associated with 1698 genes that was collected at Stanford from various sources such as Kyoto Encyclopedia of Genes and Genomes (KEGG), BioCarta, and Signaling PAthway Database (SPAD). The pathways were expressed as an Excel™ spreadsheet with each row representing a pathway name followed by the symbols for the pathway members.

Another major source of protein interaction information that is being widely researched is scientific text. To expand the list of gene symbols in each pathway in the database a prototype scientific literature association tool is used. Briefly, a large association network (an association represents a relation among genes or proteins extracted from a sentence of text) is automatically constructed using BioFerret and ALFA (Vailaya et al., 2005, Bioinformatics, 21:430-438) from over 250,000 PubMed abstracts. An extended pathway is constructed for each pathway in the database of 360 pathways as follows. For every gene symbol in a given pathway, all genes not in the pathway but associated with this gene via the literature association network were added to the extended pathway. The total number of 1357 genes was added to the individual pathways through the literature association network. The microarray data is then analyzed in terms of both the curated pathways and the extended pathways as follows. Given a subset of differentially expressed genes, 1, and a list of pathways (each represented in terms of its genes), a statistical score is computed for each pathway, similar to the GO analysis. Since the pathway analysis is performed as part of an interactive user-guided process, we utilized the more computationally efficient Z scores instead of P values for the pathway analysis. Similar to Doniger et al., the score is calculated in terms of the statistical over- or under-occurrence of the genes from list 1 in each pathway.

$$Z ( p , l ) = \frac{( k - K \frac{n}{N} )}{\sqrt{K ( \frac{n}{N} ) ( 1 - \frac{n}{N} ) ( 1 - \frac{K - 1}{N - 1} )}} ,$$

where N=the total number of unique genes represented in the microarray, n=the total number of unique genes represented in the set, 1, K=the total number of entries in N mapping to a specific pathway, p, and k=the number of entries in n mapping to a specific pathway.

The Z-score represents a surprise in finding “k” terms when “nK/N” terms are expected. A high positive value (signifying statistical over-abundance) or high negative value (signifying statistical under-abundance) of Z-score for a pathway implies a significant surprise level, and hence, the greater representation of the pathway based on the experimental data. A high Z score signified statistical over-abundance of genes in a specific pathway. A significant pathway was defined as Z scores of up regulated (top) or down-regulated (bottom) genes greater than 3.0 and with three or more genes associated the scientific literature. Based on the above-defined methods, each pathway can be statistically scored for significance under different experimental conditions.

However, associations extracted automatically from scientific text can be inherently noisy. Almost every gene could be related to every other gene by a few sets of genes and associations. These genes are either regulators of very broadly defined processes or have been extensively reported in the literature. Nevertheless, this statistical approach reduces the noise effect since it discriminates against genes or terms that occur in a large number of pathways. To be consistent with GO analysis, the Z score is converted to P-value. The number of gene counts for each significant PA term is listed in, for example, Table 2.

GO and PA terms with P value <0.005 are considered significant. Optionally, to explore the impact of multiple testing correction, Bonferroni correction to the P-values is applied. For example, see Tables 1-2. Bonferroni correction assumes independence of the tests performed (Osier et al., 2004, BMC Bioinformatics. 5:124) and, given the complex dependency structure between terms in the GO hierarchy, Bonferroni adjusted P-values may be potentially overly conservative (Slonim, 2002, Nat Genet. 32 Suppl: 502-508)

### Therapeutic Targets and Diagnostic Markers

### Diagnosis

One aspect of the invention, provides for a method for diagnosing a patient or subject having or developing cardiovascular disease comprising detecting a change in expression of at least one of the identified genes that are differentially expressed in coronary artery endothelial cells and/or smooth muscle cells. In some embodiments, the endothelial cells or smooth muscle cells have been exposed to an atherogenic agent. Atherogenic agents can include oxLDL, IL1β, TNFα, and PDGF. The genes showing differential expression in endothelial cells and smooth muscle cells in the presence of an atherogenic agent are compared to either endothelial or smooth muscle cells in the absence of the agent and/or to saphenous vein cells similarly treated.

A method for diagnosis of cardiovascular disease or susceptiblity to cardiovascular disease comprises detecting the expression of one or more genes, for example, as listed in Tables 3-4, 23-24, and 32 or 33 in a biological sample, and determining whether the level of expression of the gene matches the level of expression of the gene in a endothelial cell and/or smooth muscle cell treated with an atherogenic agent. In some embodiments, the expression of at least two, three, four, five, six, seven, eight, nine, ten or all of the genes shown in Table 3-4, 23-24, 32, or 33 are detected. In some embodiments, the genes are upregulated or downregulated at least 1.5 fold as compared to untreated cells under the same conditions. In other embodiments, the genes are upregulated or downregulated at least about 3 fold and more preferably 10 fold. In other embodiments, the genes that are identified for diagnostic or therapeutic purposes are those that are upregulated or downregulated at least 3 fold compared to control with at least a P value <0.0001, preferably a P value up to 10^{-8.}

In some embodiments, the biological sample is serum or blood. In other embodiments, the biological sample is coronary artery smooth muscle cells or endothelial cells.

In some embodiments, the expression of the genes is detected by detecting the polypeptide. Sequence of polypeptides encoded by the genes can be determined using publicly accessible databases and the gene names. In some embodiments, the polypeptide is a secreted polypeptide and may be measured in blood or serum. In some embodiments, the expression of the polypeptide is increased at least 1.5 fold, more preferably about 5 fold and more preferably about 10 fold or more above untreated cells under the same conditions. In other embodiments, the expression of the polypeptide is decreased about 1.5 fold, more preferably 3 fold and more preferably, 10 fold.

In some embodiments, expression of the polypeptide is detected using antibodies that specifically bind to the polypeptide. In some embodiments, the antibody is detectably labelled. Labels can include, for example, green fluorescent protein, enzymes, biotin, fluorescent moieties, and radioactive moieties. In other embodiments, an activity assay might be utilized to detect the polypeptide. Such activity assays depend on the nature of the polypeptide and many are known in the art and may be described below.

In other embodiments, expression of a gene may be detected with a primer or probe that specifically recognizes the gene. Such probes have been described and identified on the array such as those of Agilent. Alternatively, other databases might provide for cloned sequences that can detect a particular gene. Primers can be designed to amplify a particular sequence in accord with methods known in the art and can be utilized in PCR methods such as real time PCR.

Several types of genes have been identified as being associated with coronary artery smooth muscle cells and endothelial cells that have been treated with an atherogenic agent. The expression profile of one or more of these genes can be utilized to diagnose cardiovascular disease or susceptibility to cardiovascular disease. Some of these genes are described in more detail below.

### Insulin Growth Factor Binding Proteins (IGFBPs)

OxLDL and PDGF differentially regulate several insulin growth factor binding protein (IGFBPs) in coronary artery smooth muscle cells (CASM) as compared to treated saphenous vein smooth muscle cells (SVSM). IGFBP2, IGFBP3, IGFBP4, IGFBP5, IGFBP6, and IGFBP7 are all upregulated in response to OxLDL in CASMC. Specifically, IGFBP2, IGFBP3, IGFBP4, IGGBP5, and IGFBP6 are upregulated 4.2-, 1.5-, 2.6-, 1.6-, and 2.9-fold, respectively. However, IGFBP3, IGFBP4, and IGFBP7 are downregulated 1.56-, 1.63-, and 1.98-fold, respectively, in response to PDGF in CASMC.

IGFBPs bind insulin growth factors (IGFs) and act as carriers for IGFs. IGFBPs can inhibit IGF-1 action, although IGFBP-1, -3, and -5 have been shown to stimulate select IGF-1 activities (Delafontaine et al., 2004, Aterioscler. Thromb. Vasc. Biol., 24: 1-10). IGFBPs are not usually present in detectable levels in healthy coronary arteries, yet are present in markedly increased levels in atherectomy samples (Grant et al., 1996, Regul. Pept. 67: 137-144).

A polynucleotide and amino acid sequence for IGFBP2 are provided in P18065
(GI:124058). A polynucleotide and amino acid sequence for IGFBP3 are provided in P17936
(GI:124062). A polynucleotide and amino acid sequence for IGFBP4 are provided in P22692
(GI:124065). A polynucleotide and amino acid sequence for IGFBP5 are provided in P24593
(GI:124069). A polynucleotide and amino acid sequence for IGFBP6 are provided in P24592
(GI:124068). A polynucleotide and amino acid sequence for IGFBP7 are provided in AAH17201.1 (GI:16877961).

In some embodiments, a method for diagnosing cardiovascular disease comprises: detecting at least one of a polypeptide selected from the group consisting of IGFBP2, IGFBP3, IGFBP4, IBFBP5, IGFBP6, IGFBP7 and mixtures thereof, in a biological sample, wherein an increase in IGFBP2, IGFBP3, IGFBP4, IBFBP5, IGFBP6, and/or IGFBP7 polypeptide as compared to control sample (a same cell type or tissue in a healthy state) is indicative of cardiovascular disease or susceptibility to cardiovascular disease. In some embodiments, the IGFBP polypeptide is detected using an antibody. In other embodiments, the IGFBP polypeptide is detected by an activity assay. Activity assays for IGFBP are known to those of skill in the art and include binding to IGF-1.

In some embodiments, the method provides for diagnosis of a cardiovascular condition or susceptibility to the condition selected from the group consisting of atherosclerosis, stroke, ischemic heart disease, hypertension, cardiac hypertrophy, post-angioplasty restenosis, angina, and coronary heart disease.

In some embodiments, a method may comprise detecting expression of a gene encoding a IGFBP2, IGFBP3, IGFBP4, IGFBP5, IGFBP6, IGFBP7, or mixtures thereof, polypeptide in a biological sample, wherein an increase in expression of the gene is indicative of cardiovascular disease or susceptiblity to cardiovascular disease.

In the method, expression of the gene may be detected using a probe and/or a primer. Probes for detecting genes are known to those of skill in the art and are commercially available, for example, from Agilent. Alternatively, other nucleic acid sequences can be used to detect a gene as described in publicly available databases. Primers can be designed in methods known in the art and can be utilized in PCR methods such as realtime PCR.

### Chemokines

OxLDL and PDGF differentially regulate several chemokines in coronary artery smooth muscle cells (CASMC) as compared to treated saphenous vein smooth muscle cells (SVSMC). For example, chemokine CXCL12 is dramatically upregulated by OxLDL (44-fold) and PDGF in CASM, whereas in SVSM it was not changed by OxLDL but downregulated by PDGF. Both OxLDL and PDGF downregulate the expression of CXCL10 and CXCL11.

Chemokines are secreted proteins and as such, may be measured in biological samples such as serum or blood as well as in cells such aortic smooth muscle cells. A polynucleotide and amino acid sequence for CXCL12 are provided in NM_{—}199168 (GI:76563931). A polynucleotide and amino acid sequence for CCL13 are provided in NM₁₃ 005408 (GI: 22538799). A polynucleotide and amino acid sequence for CXCL10 are provided in NM_{—}001565 (4504700). A polynucleotide and amino acid sequence for CXCL11 are provided in NM_{—}005409 (GI:14790145).

In some embodiments, a method for diagnosing cardiovascular disease comprises: detecting a CXCL12 and/or CCL13 polypeptide in a biological sample, wherein an increase in CXCL12 and/or CCL13 polypeptide as compared to control sample (a same cell type or tissue in a healthy state) is indicative of cardiovascular disease or susceptibility to cardiovascular disease. In some embodiments, the CXCL12 or CCL13 polypeptide is detected using an antibody. In other embodiments, the polypeptide is detected by binding to its receptor CXCR4, for example, a CXCR4 receptor having a sequence as provided in NM_{—}003467 (GI:56790928). In other embodiments, the CXCL12 polypeptide is detected by an activity assay. Activity assays for CXCL12 or CCL13 are known to those of skill in the art and include chemotactic activity for lymphocytes, and cleavage by matrix metalloproteinase-2.

In some embodiments, the method provides for diagnosis of a cardiovascular condition or susceptibility to the condition selected from the group consisting of atherosclerosis, stroke, ischemic heart disease, hypertension, cardiac hypertrophy, post-angioplasty restenosis, angina, and coronary heart disease.

In some embodiments, the method further comprises detecting a CXCL11 or CXCL10 polypeptide or both, wherein a decrease in the CXCL11 or CXCL10 polypeptide is indicative of cardiovascular disease. In some embodiments, CXCL11 or CXCL10 polypeptide, or both, are detected with a specific antibody that binds to CXCL11 or CXCL10, respectively.

In some embodiments, a method may comprise detecting expression of a gene encoding a CXCL12 and/or CCL13 polypeptide in a biological sample, wherein an increase in expression of the gene is indicative of cardiovascular disease or susceptiblity to cardiovascular disease. In some embodiments, the method further comprises detecting expression of a gene encoding a CXCL11 or CXCL10 polypeptide or both, wherein a decrease in the expression of the gene encoding a CXCL11 or CXCL10 polypeptide is indicative of cardiovascular disease.

In the method, expression of the gene may be detected using a probe and/or a primer. Probes for detecting genes are known to those of skill in the art and are commercially available, for example, from Agilent. Alternatively, other nucleic acid sequences can be used to detect a gene as described in publicly available databases. Primers can be designed in methods known in the art and can be utilized in PCR methods such as realtime PCR.

### Extracellular Matrix (ECM)

Genes from several ECM gene families are differentially expressed between CASM and SVSM in response to OxLDL and PDGF. Among MMP genes, MMP2 is the isoform most significantly upregulated in CASM, while MMP10 was downregulated. Also, OxLDL and PDGF induce TIMP1 and TIMP2 expression in CASM but not in SVSM. OxLDL induce differential regulation of collagen (COL) and integrin genes, the major receptors for ECM-mediated cell adhesion, migration, proliferation and differentiation in CASM but had effects on only a few of these genes in SVSM. Interestingly, decorin and fibronectin, two important ECM genes that negatively regulate cell growth, are significantly upregulated in CASM but downregulated in SVSM, which is consistent with the SMC proliferative and migratory responses.

A polynucleotide and amino acid sequence for MMP2 are provided in NM_{—}032549 (GI:14211844) A polynucleotide and amino acid sequence for MMP10 are provided in NM_{—}002425 (GI:4505204). A polynucleotide and amino acid sequence for TIMP1 are provided in NM_{—}003254 (GI:73858576). A polynucleotide and amino acid sequence for TIMP2 are provided in NM_{—}003255 (GI:73858577). A polynucleotide and amino acid sequence for decorin are provided in NM_{—}001920 (GI:47419925). A polynucleotide and amino acid sequence for fibronectin are provided in NM_{—}014923 (GI:39930340).

In some embodiments, a method for diagnosing cardiovascular disease comprises: detecting at least one, preferably at least two or more ECM polypeptides of Table 16 in a biological sample, wherein an increase in ECM polypeptide as compared to control sample (a same cell type or tissue in a healthy state) is indicative of cardiovascular disease or susceptibility to cardiovascular disease. In some embodiments, the ECM polypeptide is selected from the group consisting of MMP2, TIMP1, TIMP2, COL1A2, COL3A1, COL6A1, COL12A1, COL16A1, COL13A1, COL15A1, COL6A3, COL9A3, COL4A4, COL1A1, ITGA4, ITGBL, ITGB5, ITGB1, ITGA9, ITGA2B, decorin and fibronectin, and mixtures thereof. In some embodiments, the polypeptide is detected using an antibody. In other embodiments, polypeptide is detected by an activity assay. Activity assays for ECM are known to those of skill in the art.

In some embodiments, the method provides for diagnosis of a cardiovascular condition or susceptibility to the condition selected from the group consisting of atherosclerosis, stroke, ischemic heart disease, hypertension, cardiac hypertrophy, post-angioplasty restenosis, angina, and coronary heart disease.

In some embodiments, the method further comprises detecting a MMP10, MMP3 and/or MMP20 polypeptide, wherein a decrease in the MMP10, MMP3 and/or MMP20 polypeptide is indicative of cardiovascular disease. In some embodiments, the MMP10 polypeptide is detected with a specific antibody that binds to MMP10.

In some embodiments, a method may comprise detecting expression of at least one gene encoding an ECM polypeptide of Table 16 in a biological sample, wherein an increase in expression of the gene is indicative of cardiovascular disease or susceptiblity to cardiovascular disease. In some embodiments, the gene encodes a polypeptide selected from the group consisting of MMP2, TIMP1, TIMP2, COL1A2, COL3A1, COL6A1, COL12A1, COL16A1, COL13A1, COL15A1, COL6A3, COL9A3, COL4A4, COL1A1, ITGA4, ITGBL, ITGB5, ITGB1, ITGA9, ITGA2B, decorin and fibronectin, and mixtures thereof. In some embodiments, the method further comprises detecting expression of a gene encoding a MMP10, MMP3 and/or MMP20 polypeptide, wherein a decrease in the expression of the gene encoding a MMP10, MMP3 and/or MMP20 polypeptide is indicative of cardiovascular disease.

In the method, expression of the gene may be detected using a probe and/or a primer. Probes for detecting genes are known to those of skill in the art and are commercially available, for example, from Agilent. Alternatively, other nucleic acid sequences can be used to detect a gene as described in publicly available databases. Primers can be designed in methods known in the art and can be utilized in PCR methods, such as real time PCR.

### Endothelial and Smooth Muscle Cell Marker Genes

In CASM but not in SVSM, OxLDL significantly (P<0.001) suppresses (by 1.7-44 fold) several endothelial cell (EC) marker genes including EDN1 (endothelin 1), VCAM1, PECAM1, CDH5, MMRN1, ESM1, LIPG, THBD and C1QR1. EDN receptor type B (EDNRB) is upregulated by OxLDL (Table 18). OxLDL in CASM upregulates several smooth muscle genes including SM22-α, SM α-actin, vimentin, and ADD3, but not the cardiac muscle actin-α.

A polynucleotide and amino acid sequence for Transgelin (SM22-alpha) are provided in Q01995 and NM_{—}003186 (GI:48255904). A polynucleotide and amino acid sequence for alpha actin 2 are provided in P03996 (GI:113266). A polynucleotide and amino acid sequence for adducin 3 (gamma) are provided in Q9UEY8 and NM_{—}016824 (GI:62912451). A polynucleotide and amino acid sequence for Vimentin are provided in P08670 and NM_{—}003380 (GI:62414288). A polynucleotide and amino acid sequence for EDN receptor type B (EDNRB) are provided in NM_{—}003991.1 (GI:4503466).

A polynucleotide and amino acid sequence for endothelin 1 (END 1) are provided in NM_{—}001955 (GI:21359861). A polynucleotide and amino acid sequence for vascular cell adhesion molecule 1 (VCAM1) are provided in NM_{—}001078 (GI:18201907). A polynucleotide and amino acid sequence for CD31 (PECAM 1) are provided in NM_{—}000442 (GI:21314616). A polynucleotide and amino acid sequence for vascular endothelial cadherin (CDH5) are provided in AB035304 (GI:6483307). A polynucleotide and amino acid sequence for multimerin 1 (MMRN1) are provided in NM_{—}007351 and (GI:45269140). A polynucleotide and amino acid sequence for endothelial cell specific molecule (ESM1) are provided in NM_{—}007036 (GI:13259505). A polynucleotide and amino acid sequence for endothelial lipase (LIPG) are provided in NM_{—}006033 (GI:62422575). A polynucleotide and amino acid sequence for thrombomodulin (THBD) are provided in NM_{—}000361 and (GI:40288292). A polynucleotide and amino acid sequence for complement C1q receptor 1 (C1QR1) are provided in Q9NPY3 (GI:8247033).

In some embodiments, a method for diagnosing cardiovascular disease comprises: detecting at least one, preferably at least two or more smooth muscle polypeptides of Table 18 in a biological sample, wherein an increase in smooth muscle polypeptide as compared to control sample (a same cell type or tissue in a healthy state) is indicative of cardiovascular disease or susceptibility to cardiovascular disease. In some embodiments, the smooth muscle polypeptide is selected from the group consisting of EDN receptor type B (EDNRB), transgelin, alpha actin 2, adducin 3, vimentin, and mixtures thereof. In some embodiments, the polypeptide is detected using an antibody. In other embodiments, polypeptide is detected by an activity assay. Activity assays for smooth muscle polypeptides are known to those of skill in the art.

In some embodiments, the method provides for diagnosis of a cardiovascular condition or susceptibility to the condition selected from the group consisting of atherosclerosis, stroke, ischemic heart disease, hypertension, cardiac hypertrophy, post-angioplasty restenosis, angina, and coronary heart disease.

In some embodiments, the method further comprises detecting at least one, preferably two or more EDN1 (endothelin 1), VCAM1, PECAM1, CDH5, MMRN1, ESM1, LIPG, THBD and C1QR1 polypeptide, wherein a decrease in the EDN1 (endothelin 1), VCAM1, PECAM1, CDH5, MMRN1, ESM1, LIPG, THBD and C1QR1 polypeptide and mixtures thereof, wherein a decrease in the levels of the polypeptide is indicative of cardiovascular disease. In some embodiments, each polypeptide is detected with a specific antibody that binds to the polpeptide.

In some embodiments, a method may comprise detecting expression of at least one, preferably two or more or all, gene encoding a smooth muscle cell polypeptide of Table 18 in a biological sample, wherein an increase in expression of the gene is indicative of cardiovascular disease or susceptiblity to cardiovascular disease. In some embodiments, the gene encodes a polypeptide selected from the group consisting of EDN receptor type B (EDNRB), transgelin, alpha actin 2, adducin 3, vimentin, and mixtures thereof. In some embodiments, the method further comprises detecting expression of at least one, preferably two or more genes encoding EDN1 (endothelin 1), VCAM1, PECAM1, CDH5, MMRN1, ESM1, LIPG, THBD and C1QR1 polypeptide and mixture thereof, wherein a decrease in the expression of a gene encoding EDN1 (endothelin 1), VCAM1, PECAM1, CDH5, MMRN1, ESM1, LIPG, THBD and C1QR1 polypeptide is indicative of cardiovascular disease.

In the method, expression of the gene may be detected using a probe and/or a primer. Probes for detecting genes are known to those of skill in the art and are commercially available, for example, from Agilent. Alternatively, other nucleic acid sequences can be used to detect a gene as described in publicly available databases. Primers can be designed in methods known in the art and can be utilized in PCR methods, such as real time PCR.

### Cellular Proliferation Genes

OxLDL induces sets of genes that regulate cellular proliferation only in CAEC (endothelial cells). These genes include up-regulated genes known to promote cell growth, such as protein tyrosine kinase SYK (SYK), VGEF receptors neuropilin (NRP)1 and NRP2; and down-regulated genes that inhibit cell growth, like IGFBP3, fibroblast growth factor-inducible 14 (FN14) and IFN1β. OxLDL inhibits 8 out of 21 genes from the H1 histone family present on the arrays. Histone H1 is a linker of histone proteins involved in the condensation of chromatin. Down-regulation of H1 histone genes might decompress chromatin and facilitate cell proliferation. OxLDL up-regulates pro-apoptotic genes in CAEC, such as CASP3, TNFSF10, THBS1 and THBS4, and down-regulates anti-apoptotic genes, like TNF receptor-associated factor 4 (TRAF4), inducible T-cell co-stimulator (ICOS) and PIM1.

A polynucleotide and amino acid sequence for SYK are provided in P43405 and NM_{—}003177 (GI:34147655). A polynucleotide and amino acid sequence for NRP1 are provided in O14786 and NM_{—}003873 (GI:66912183). A polynucleotide and amino acid sequence for NRP2 are provided in AAG41897.1 and NM_{—}201266 (GI:41872561). A polynucleotide and amino acid sequence for CASP3 are provided in P42574 and NM_{—}004346 (GI:73622121). A polynucleotide and amino acid sequence for TNFSF10 are provided in P50591 and NM_{—}003810 (GI:23510439). A polynucleotide and amino acid sequence for THBS1 are provided in P07996 and NM_{—}003246 (GI:40317625). A polynucleotide and amino acid sequence for THBS4 are provided in P35443 and NM_{—}003248 (GI:40549419). A polynucleotide and amino acid sequence for TRAF4 are provided in AAH01769.1 and NM_{—}004295 (GI:22027621). A polynucleotide and amino acid sequence for ICOS are provided in BAA81129.1 and NM_{—}012092 (GI:32483379). A polynucleotide and amino acid sequence for PIM1 are provided in P11309 and NM_{—}002648 (GI:31543400).

In some embodiments, a method for diagnosing cardiovascular disease comprises: detecting at least one, preferably at least two or more cell proliferative and/or apoptotic protein polypeptides in a biological sample, wherein an increase in the polypeptide as compared to control sample (a same cell type or tissue in a healthy state) is indicative of cardiovascular disease or susceptibility to cardiovascular disease. In some embodiments, the polypeptide is selected from the group consisting of SYK (SYK), neuropilin (NRP)1, NRP2, CASP3, TNFSF10, THBS1, THBS4, and mixtures thereof. In some embodiments, the polypeptide is detected using an antibody. In other embodiments, polypeptide is detected by an activity assay. Activity assays for cell proliferation and/or apoptotic polypeptides are known to those of skill in the art.

In some embodiments, the method provides for diagnosis of a cardiovascular condition or susceptibility to the condition selected from the group consisting of atherosclerosis, stroke, ischemic heart disease, hypertension, cardiac hypertrophy, angina, and coronary heart disease.

In some embodiments, the method further comprises detecting at least one, preferably two or more polypeptides selected from the group consisting of IGFBP3, fibroblast growth factor-inducible 14 (FN14), IFN1β, TNF receptor-associated factor 4 (TRAF4), inducible T-cell co-stimulator (ICOS), PIM1, a Histone H1 polypeptide and mixtures thereof, wherein a decrease in at least one of the IGFBP3, fibroblast growth factor-inducible 14 (FN14), IFN1β, TNF receptor-associated factor 4 (TRAF4), inducible T-cell co-stimulator (ICOS), PIM1, and a Histone H1 polypeptide and mixtures thereof is indicative of cardiovascular disease. In some embodiments, each polypeptide is detected with a specific antibody that binds to the polypeptide.

In some embodiments, a method may comprise detecting expression of at least one, preferably two or more, genes encoding a cell proliferative or apoptotic polypeptide in a biological sample, wherein an increase in expression of the gene is indicative of cardiovascular disease or susceptiblity to cardiovascular disease. In some embodiments, the gene encodes a polypeptide selected from the group consisting of SYK (SYK), neuropilin (NRP)1, NRP2, CASP3, TNFSF10, THBS1, THBS4, and mixtures thereof. In some embodiments, the method further comprises detecting expression of at least one, preferably two or more genes encoding IGFBP3, fibroblast growth factor-inducible 14 (FN14), IFN1β, TNF receptor-associated factor 4 (TRAF4), inducible T-cell co-stimulator (ICOS), PIM1, a Histone H1 polypeptide and mixtures thereof, wherein a decrease in the expression of a gene encoding IGFBP3, fibroblast growth factor-inducible 14 (FN14), IFN1β, TNF receptor-associated factor 4 (TRAF4), inducible T-cell co-stimulator (ICOS), PIM1, a Histone H1 polypeptide and mixtures thereof, is indicative of cardiovascular disease.

In the method, expression of the gene may be detected using a probe and/or a primer. Probes for detecting genes are known to those of skill in the art and are commercially available, for example, from Agilent. Alternatively, other nucleic acid sequences can be used to detect a gene as described in publicly available databases. Primers can be designed in methods known in the art and can be utilized in PCR methods such as real time PCR.

### Alzheimer's Related Genes

In arterial cells, OxLDL activates pathways related to Alzheimer's disease. These genes include SYK, pyruvate dehydrogenase kinase 4 (PDK4), microtubule-associated protein 2 (MAP2), calmodulin 3 (calm3), CASP3, and aldehyde dehydrogenase 6 (ALDH1α3).

A polynucleotide and amino acid sequence for SYK are provided in NM_{—}003177 (GI:34147655). A polynucleotide and amino acid sequence for PDK4 are provided in NM_{—}002612 (GI:33589822). A polynucleotide and amino acid sequence for MAP2 are provided in NM_{—}031846 (GI:14195619). A polynucleotide and amino acid sequence for CASP3 are provided in NM_{—}004346 (GI:73622121). A polynucleotide and amino acid sequence for CALM3 are provided in NM_{—}005184 (GI:58218967). A polynucleotide and amino acid sequence for ALDH1α3 are provided in NM_{—}000693 (GI:4502040).

In some embodiments, a method for diagnosing cardiovascular disease comprises: detecting at least one, preferably at least two or more polypeptides in a biological sample, wherein an increase in the polypeptide as compared to control sample (a same cell type or tissue in a healthy state) is indicative of cardiovascular disease or susceptibility to cardiovascular disease. In some embodiments, the polypeptide is selected from the group consisting of SYK, pyruvate dehydrogenase kinase 4 (PDK4), microtubule-associated protein 2 (MAP2), calmodulin 3 (calm3), CASP3, and aldehyde dehydrogenase 6 (ALDH1α3) and mixtures thereof. In some embodiments, the polypeptide is detected using an antibody. In other embodiments, polypeptide is detected by an activity assay. Activity assays for the polypeptides are known to those of skill in the art.

In some embodiments, the method provides for diagnosis of a cardiovascular condition or susceptibility to the condition selected from the group consisting of atherosclerosis, stroke, ischemic heart disease, hypertension, cardiac hypertrophy, post-angioplasty restenosis, angina, and coronary heart disease.

In some embodiments, a method may comprise detecting expression of a gene encoding an Alzheimer's related polypeptide in a biological sample, wherein an increase in expression of the gene is indicative of cardiovascular disease or susceptiblity to cardiovascular disease. In some embodiments, the gene encodes a polypeptide selected from the group consisting of SYK, pyruvate dehydrogenase kinase 4 (PDK4), microtubule-associated protein 2 (MAP2), calmodulin 3 (calm3), CASP3, and aldehyde dehydrogenase 6 (ALDH1α3) and mixtures thereof.

In the method, expression of the gene may be detected using a probe and/or a primer. Probes for detecting genes are known to those of skill in the art and are commercially available, for example, from Agilent. Alternatively, other nucleic acid sequences can be used to detect a gene as described in publicly available databases. Primers can be designed in methods known in the art and can be utilized in PCR methods such as real time PCR.

### TNFα and IL1β Induced Genes

Treatment of coronary artery endothelial cells with TNFα and IL1β results in differential expression of a set of genes. These genes are shown in Tables 27-30. For example, TNFα up-regulated apoptosis genes, e.g. CASP1, and down-regulated anti-inflammatory genes, e.g. PAFAH2 in CAEC cells.

Polynucleotide and amino acids sequences for each of the differentially regulated genes in coronary artery endothelial cells are provided by an NCBI sequence code for each gene in the Table 20.

In some embodiments, a method for diagnosing cardiovascular disease comprises: detecting at least one, preferably at least two or more or all polypeptides of any one of Tables 20, 23, 27, or 29 in a biological sample, wherein an increase in the polypeptide as compared to control sample (a same cell type or tissue in a healthy state) is indicative of cardiovascular disease or susceptibility to cardiovascular disease. In other embodiments, three, four, five six, seven, eight nine or ten, or even all of the genes that are upregulated at least 1.5 fold in Tables 27 and 29 are detected. In some embodiments, the polypeptide is selected from the group consisting CXCL10, IL8, SERPINE1 (PAI1), E selectin (SELE), superoxide dismutase 2 (SOD2), TNFα inducible protein 6, ICAM1, VCAM1, IL6, chemokine ligand 3 (CXCL3), and mixtures thereof. In some embodiments, the polypeptide is detected using an antibody. In other embodiments, polypeptide is detected by an activity assay. Activity assays for the polypeptides are known to those of skill in the art.

In some embodiments, the method further comprises detecting at least one, preferably two or more polypeptides selected from the group consisting of Gla protein (MGP), thrombomodulin (THBD), CXC chemokine receptor 4 (CXCR4), and mixtures thereof, wherein a decrease in at least one of the Gla protein, thrombomodulin, CXC chemokine receptor 4, and mixtures thereof is indicative of cardiovascular disease. In other embodiments, three, four, five six, seven, eight, nine or ten, or even all of the genes that are downregulated at least 1.5 fold in Tables 20, 23, 27, and/or 29 are detected. In some embodiments, the polypeptide is selected from the group consisting Gla protein (MGP), thrombomodulin (THBD), CXC chemokine receptor 4 (CXCR4), and mixtures thereof. In some embodiments, each polypeptide is detected with a specific antibody that binds to the polypeptide.

In some embodiments, the method provides for diagnosis of a cardiovascular condition or susceptibility to the condition selected from the group consisting of atherosclerosis, stroke, ischemic heart disease, hypertension, cardiac hypertrophy, post-angioplasty restenosis, angina, and coronary heart disease.

In some embodiments, a method may comprise detecting expression of at least one, preferably two or more, gene encoding a TNFα or IL1β stimulated polypeptide in a biological sample, wherein an increase in expression of the gene is indicative of cardiovascular disease or susceptiblity to cardiovascular disease. In some embodiments, the gene encodes a polypeptide selected from the group consisting of CXCL10, IL8, SERPINE1 (PAI1), E selectin (SELE), superoxide dismutase 2 (SOD2), TNFα inducible protein 6, ICAM1, VCAM1, IL6, chemokine ligand 3 (CXCL3), and mixtures thereof. In some embodiments, the method further comprises detecting expression of at least one, preferably two or more genes encoding Gla protein (MGP), thrombomodulin (THBD), CXC chemokine receptor 4 (CXCR4), and mixtures thereof, wherein a decrease in the expression of a gene encoding Gla protein (MGP), thrombomodulin (THBD), CXC chemokine receptor 4 (CXCR4), and mixtures thereof, is indicative of cardiovascular disease.

A polynucleotide and amino acid sequence for IL8 are provided in NM_{—}000584.2 (GI:28610153). A polynucleotide and amino acid sequence for IL6 are provided in NM_{—}000600.1 (GI:10834983). A polynucleotide and amino acid sequence for SERPINE 1 (PAI1) are provided in NP_{—}000593 (GI:10835159). A polynucleotide and amino acid sequence for E-selectin (SELE) are provided in NM_{—}000450.1 (GI:4506870). A polynucleotide and amino acid sequence for superoxide dismutase 2 (SOD2) are provided in NM_{—}000636.1 (GI:10835186). A polynucleotide and amino acid sequence for intercellular adhesion molecule 1 (ICAM1) are provided in NM_{—}000201.1 (GI:4557877). A polynucleotide and amino acid sequence for vascular cellular adhesion molecule (VCAM) are provided in NM_{—}001078.2 (GI:18201907). A polynucleotide and amino acid sequence for chemokine ligand 3 (CXCL3) are provided in NM_{—}002090.1 (GI:4504156).

A polynucleotide and amino acid sequence for Gla protein (MGP) are provided in NM_{—}000900.1 (GI:4505178). A polynucleotide and amino acid sequence for thrombomodulin) are provided in NM_{—}000361 (GI:40288292). A polynucleotide and amino acid sequence for CXC chemokine receptor 4 (CXCR4) are provided in NM_{—}003467.1 (GI:4503174).

In the method, expression of the gene may be detected using a probe and/or a primer. Probes for detecting genes are known to those of skill in the art and are commercially available, for example, from Agilent. Alternatively, other nucleic acid sequences can be used to detect a gene as described in publicly available databases. Primers can be designed in methods known in the art and can be utilized in PCR methods, such as real time PCR.

In some embodiments, a method for diagnosing cardiovascular disease or susceptibility to cardiovascular disease comprises detecting one or more or preferably all of the genes or polypeptides encoded by the genes that are upregulated at least 1.5 fold in coronary artery smooth muscle cells or endothelial cells in response to an atherogenic stimuli. In some embodiments, the genes and/or polypeptides that are detected are those that are upregulated in comparison to control cells with a P value less than 0.0001, more preferably less than 10⁻⁸. The genes that are upregulated at least 3 fold and have a P value less than 10⁻⁸ are shown in Table 32 or 33. The upregulated genes are also shown in Tables 20, 23, 27, and/or 29. In some embodiments, the atherogenic stimulus is selected from the group consisting of OxLDL, TNFα, IL1β, and PDGF. In some embodiments, the genes include chemokines, endothelial and smooth muscle markers, cell proliferation and apoptotic gene, Alzheimer's related genes, TNFα stimulated genes, and IL1β stimulated genes as described above.

In other embodiments, the method further comprises detecting one or more or preferably all of the genes or polypeptides encoded by the genes that are down regulated at least 1.5 fold in coronary artery smooth muscle cells or endothelial cells in response to an atherogenic stimuli. In some embodiments, the genes and/or polypeptides that are detected are those that are downregulated in comparison to control cells with a P value less than 0.0001, more preferably less than 10⁻⁸. The genes that are downregulated at least 3 fold and have a P value less than 10⁻⁸ are shown in Tables 32 or 33. The downregulated genes are also shown in Tables 20, 23, 27, and/or 29. In some embodiments, the atherogenic stimulus is selected from the group consisting of oxLDL, TNFα, IL1β and PDGF. In some embodiments, the genes include chemokines, endothelial and smooth muscle markers, cell proliferation and apoptotic genes, Alzheimer's related genes, TNFα stimulated genes, and IL1β stimulated genes as described above.

The set of genes and probes or primers that can detect these genes or polynucleotides can be used to prepare a microarray, hybridization assay, PCR assay that can be used to analyze a biological sample in order to provide a diagnosis of cardiovascular disease. In some embodiments, gene products, such as polypeptides, can be detected using standard methodologies such as ELISA, immunoPCR and the like. An amino acid sequence of the polypeptides encoded by the polynucleotide are available by accessing the Accession Nos. using a publicly available database, such as the source database at Stanford.

Another aspect of the invention provides a kit for diagnosing a cardiovascular disease or susceptiblity to cardiovacular disease in a patient. A kit comprises an agent for detecting expression of at least two genes or polynucleotides, or the complements thereof, selected from the group consisting of CXCL12, CCL13 CXCL10, CXCL11 and mixtures thereof, or the complements thereof, and optionally, instructions for detecting increased expression as compared to a control, wherein enhanced expression of CXCL12 and/or CCL13 is indicative of cardiovascular disease. The control can be prepared from one or more cells including at least one housekeeping gene, or it can be the expression level of the gene in normal or healthy tissue sample.

In some embodiments, the kit comprises a plurality of agents that can detect expression of at least two or more, or all the genes or polynucleotides of Table 3, Table 23, Table 32, or Table 33 or the complements thereof that are upregulated or down regulated at least 1.5 fold in coronary artery cells treated with an atherogenic stimulus. The plurality of agents may comprise a primer or probe that can detect expression of each of the polynucleotides or genes, or their complements, of Tables 3, 23, 32, or 33. Another embodiment includes a plurality of polynucleotides comprising two or more genes or polynucleotides of Tables 3, 23, 32, or 33 or their complements, optionally attached to a solid substrate. The agents may be attached to a solid substrate such as a polystyrene plate or glass slide. Sequences of oligonucleotides or cDNA probes that can detect the genes are available from the manufacturer such as Agilent or in publicly available databases, such as the Unigene database and Image Consortium.

### Methods of Targeting a Gene Product to Produce a Therapeutic Agent Useful to Treat Cardiovascular Disease.

One aspect of the invention includes a method of targeting a product of at least one of the genes in tables 3, 23, 32, or 33 that includes identifying a therapeutic agent having a therapeutic effect on said gene product. Another embodiment includes a method of targeting a product of at least one of the genes in Tables 20, 27, and 29 for identification of an antagonist or agonist that can be utilized to treat cardiovascular disease.

Another aspect of the invention involves a method of screening for an agent that modulates a gene or polynucleotide for treatment for cardiovascular disease. A screening method comprises a method for detecting an agent that can modulate the expression of at least one gene or polynucleotide for which a change in expression is is seen in a cardaic aortic cell treated with an atherogenic agent. In some embodiments, the genes or polynucleotides are selected from the genes of Tables 3, 23, 32, or 33. In other embodiments, the genes are selected from those that have at least 3 fold upregulation or down regulation, more preferably have a P value less than 0.0001, more preferably less than 10⁻⁸.

If a gene is upregulated, a method comprises identifying an antagonist of the gene or polynucleotide. In some embodiments, a method for identifying an antagonist comprises contacting one or more or preferably all of the genes or polypeptides encoded by the genes that are upregulated at least 1.5 fold in coronary artery smooth muscle cells or endothelial cells in response to an atherogenic stimuli with a candidate molecule and determining whether the candidate molecule inhibits expression or activity of the gene or polypeptide. The upregulated genes are shown in Tables 3, 5, 20, 23, 27, 29, 32 and 33. In some embodiments, the atherogenic stimulus is selected from the group consisting of oxLDL, TNFα, IL1β and PDGF. In some embodiments, the genes include chemokines, endothelial and smooth muscle markers, cell proliferation and apoptotic genes, Alzheimer's related genes, TNFα stimulated genes, and IL1β stimulated genes as described above.

If a gene is downregulated, a method comprises identifying an agonist of the gene or polynucleotide. In some embodiments, a method for identifying an agonist comprises contacting one or more or preferably all of the genes or polypeptides encoded by the genes that are downregulated at least 1.5 fold in coronary artery smooth muscle cells or endothelial cells in response to an atherogenic stimuli with a candidate molecule and determining whether the candidate molecule increases expression or activity of the gene or polypeptide. The downregulated genes are shown in Tables 3, 5, 20, 23, 27, 29, 32, and 33. In some embodiments, the atherogenic stimuli is selected from the group consisting of OxLDL, TNFα, IL1β and PDGF. In some embodiments, the genes include chemokines, endothelial and smooth muscle markers, cell proliferation and apoptotic genes, Alzheimer's related genes, TNFα stimulated genes, and IL1β stimulated genes as described above.

Exemplary sequences for the genes, polynucleotides, or polypeptides of can be found in Accession Nos. as provided in Tables 3, 5, 20, 23, 27, 29, 31, 32 and 33. Complementary sequences for the genes and polynucleotides can readily be determined by one of skill in the art.

In an embodiment, a method for screening for an antagonist of CXCL12 comprises a) contacting a CXCL12 polypeptide with an agent; and b) determining whether the agent inhibits binding of CXCL12 to its receptor CXCR4 or inhibits an activity of the CXCL12. In some embodiments, the agent is an antibody or an aptamer. In some embodiments, the activity of CXCL12 is chemoattraction of lymphocytes or is cleavage of CXCL12 by matrix metalloproteinase-2.

In another embodiment, a method for screening for an antagonist of CXCL12 comprises contacting polynucleotide encoding a CXCL12 polypeptide, with a candidate agent, and determining whether the candidate agent inhibits expression of the polypeptide. In some embodiments, the agent comprises an antisense molecule, siRNA, or peptide nucleic acid. In some embodiments, expression of the polynucleotide is detected with a probe or with a primer and PCR.

An antagonist or agonist useful as a therapeutic agent can comprise a biological or chemical entity that is based on some aspect of a gene. Examples of therapeutic agents include, but are not limited to, vaccines, antibodies, oligonucleotide, DNA antisense, RNAi, peptide nucleic acids, aptamers, antibodies, chemical molecules, proteins, inhibitors, antagonists, or combinations thereof. Having a therapeutic effect on a gene product can include, but is not limited to, inhibition of some activity or process of a cell, cessation of some activity or process of a cell, an increase in some activity or process of a cell, interference with some process or activity of a cell, modification of the expression of at least one gene, modification of the expression of at least one gene product, modification of the function of at least one gene, and modification of the function of at least one gene product.

An antagonist of any of the genes, polynucleotides or gene products is effective to inhibit expression or activity of the gene or gene product and can include antisense nucleic acid, peptide nucleic acids, nucleic acid or protein vaccines, siRNA, aptamers, and antagonist antibodies (including humanized antibodies).

An agonist of any of the genes, polynucleotides or gene products is effective to enhance or increase expression or activity of the gene or gene products and can include agonist antibodies (including humanized antibodies). Other agonists include polynucleotides providing for expression, preferably overexpression, of the downregulated gene or polynucleotide.

Antibodies can be prepared by methods known to those of skill in the art and in references such as U.S. Pat. No. 6,331,415; Kohler et al., Eur. J Immun., 6:511 (1976); Winter et al., Ann. Rev. of Immunol., 12:433 (1994); and U.S. Pat. No. 5,225,539. In a hybridoma method, a mouse or other appropriate host animal, such as a hamster, is immunized to elicit lymphocytes that produce or are capable of producing antibodies that specifically bind the protein(s) used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies. Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

A peptide or polypeptide can be directly used as an immunogen to generate antibodies. Antibody is raised against antigen derived from a first mammalian species. Preferably the first mammalian species is human. However, other mammals are contemplated such as farm, pet, or zoo animals, e.g. where the antibody is intended to be used to treat such mammals. An antigen to be used for production of antibodies can be, for example, a soluble form of the full length polypeptide or a fragment thereof, such as a solubilized full length molecule or a fragment.

The antagonists and/or agonists identified herein may be utilized in methods to treat cardiovascular disease. For example, a therapeutic agent such as a humanized antibody or antisense nucleic acid may be administered to a patient in order to downregulate expression of genes or polynucleotides that are upregulated in coronary artery cells. Such therapeutic agents may be utilitized in combination with other therapies, including conventional chemotherapeutic agents.

Antibody Fragments. In certain circumstances it may be advantageous to use antibody fragments, rather than whole antibodies in therapeutic treatments. The smaller size of the fragments allows for rapid clearance, and may lead to improved access to solid tumors, for example. These antibody fragments can be therapeutic products having related variable domain framework regions to be recognized by a pan-specific anti-hzAbAb.

Various techniques have been developed for the production of antibody fragments. Traditionally, fragments are derived via proteolytic digestion of intact antibodies (see, e.g., Morimoto et al., 1992, Journal of Biochemical and Biophysical Methods 24:107-117; and Brennan et al., 1985, Science, 229:81). However, fragments can now be produced directly by recombinant host cells. Fab, Fv and ScFv antibody fragments can all be expressed in and secreted from E. coli, allowing the facile production of large amounts of these fragments. Alternatively, Fab′-SH fragments can be directly recovered from E. coli and chemically coupled to form F(ab′)₂ fragments (Carter et al., 1992, Bio/Technology 10:163-167). In another approach, F(ab′)₂ fragments can be isolated directly from recombinant host cell culture. Fab and F(ab′)₂ fragments with increased in vivo half-life comprising a salvage receptor binding epitope residues are described in U.S. Pat. No. 5,869,046. Other techniques for the production of antibody fragments will be apparent to the skilled practitioner. An antibody of choice can be a single chain Fv fragment (scFv). See WO 93/16185; U.S. Pat. No. 5,571,894; and U.S. Pat. No. 5,587,458. Fv and sFv are the only species of fragments with intact combining sites that are devoid of constant regions; thus, and are suitable for reduced nonspecific binding during in vivo use. sFv fusion proteins may be constructed to yield fusion of an effector protein at either the amino or the carboxy terminus of an sFv. See Antibody Engineering, ed. Borrebaeck. The antibody fragment may also be a “linear antibody”, e.g., as described in U.S. Pat. 5,641,870 for example. Such linear antibody fragments may be monospecific or bispecific.

Polyclonal antibodies. Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, and the like.

Animals can be immunized against an antigen, an antigen cocktail, immunogenic conjugates, or derivatives by combining, for example, 100 μg or 5 μg of the protein or conjugate (for rabbits or mice, respectively) with 3 volumes of Freund's complete adjuvant and injecting the solution intradermally at multiple sites. One month later the animals are boosted with 1/5 to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

### Selecting Targets Molecules for Therapeutic or Diagnostic Applications

Another aspect of the invention involves selecting one or more target genes or polypeptides as a therapeutic target or diagnostic marker. In a gene expression profile, many genes are upregulated or down regulated and in order to identify some of the best candidates for development for therapy or diagnostics, additional data analysis can be conducted. This data analysis includes Gene ontology (GO) and pathway analysis (PA) as described here in. In an embodiment, the method comprises selecting at least one gene or polypeptide that is upregulated or downregulated at least 1.5 fold in coronary artery cells treated with a stimuli by determining whether the gene is a member of a category of genes in both a gene ontology and pathway analysis. In some embodiments, the category of genes is selected from from the group consisting of cell proliferation genes, apoptotic genes, genes encoding IGFBPs, insulin-like growth factor signaling pathway genes, integrin signaling pathway, chemokine genes, and ECM genes.

Analysis of the gene expression data for gene ontology and pathway analysis has been described elsewhere herein. For example, by analyzing GO and PA terms, pathways significantly over-represented by OxLDL regulated genes in CAEC but not in SVEC can be identified. See Table 22. For example, cell adhesion pathways are not significantly up-regulated in SVEC but were in CAEC, consistent with the previous report that OxLDL does not induce adhesion molecule expression in SVEC, but does in arterial ECs. Regulation of focal adhesion and inflammatory response in SVEC are associated with OxLDL-induced down-regulated genes. Similarly, genes down-regulated by OxLDL in SVEC were significantly associated with pathways known to be important in atherosclerosis, such as “induction of apoptosis,” the “NF-KB pathway” and “regulation of cell cycle”.

In arterial cells, OxLDL activated pathways related to Alzheimer's disease and Huntington's Disease. See Table 26. These genes include SYK, pyruvate dehydrogenase kinase 4 (PDK4), microtubule-associated protein 2 (MAP2), calmodulin 3 (calm3), CASP3, and aldehyde dehydrogenase 6 (ALDH1α3). The potential link between Alzheimer's disease and atherosclerosis has been recently suggested in a mouse model.

GO term analysis yields a similar pattern of significant terms for IL1β and TNFα in both CAEC and SVEC, including pathways known to be associated with TNFα and IL1β. These terms included “inflammatory response,” “apoptosis,” “cell proliferation,” “immune response” and “cytokine/chemokine”. Similarly, PA term analysis found many pathways known to be associated with TNFα were significantly over-represented, including “apoptosis”, “cytokine and inflammatory response,” “NF-κb signaling,” “toll like receptor,” “IL6, IL1 and IFN signaling” pathways For example, that TNFα- and IL1β-activated anti-apoptosis pathways in SVEC are clearly identified by GO analysis while TNFα- and IL1β-activated pathways related to Huntington's disease in CAEC were more evident in PA analysis.

For cardiac smooth muscle cells, GO analysis identifies gene families of greater interest for target development as shown in Table 9. These gene families include intracellular matrix, protease inhibitor, collagen, cell proliferation, cell growth, integrin mediated signaling pathway, insulin-like growth factor signaling and binding, hemostasis, cell matrix adhesion, antioxidants, and calcium ion binding.

References cited within this application, including patents, published applications and other publications, are hereby incorporated by reference.

### EXAMPLES

The present invention may be better understood with reference to the following examples. These examples are intended to be representative of specific embodiments of the invention, and are not intended as limiting the scope of the invention.

### Example 1

### Cell Proliferation and Migration

### Methods

Cell Culture. Primary cultures of human coronary artery endothelial cells (CAEC), human saphenous vein endothelial cells (SVEC), human coronary artery smooth muscle cells (CASMC), and human saphenous vein smooth muscle cells (SVSMC) were obtained from Cambrex Bio Science Walkersville, Inc. (Walkersville, Md.). All cells were obtained cryopreserved at third passage.

Smooth muscle cells tested positive for smooth muscle cell markers (e.g., α-actin) and negative for other non-smooth muscle cell markers (e.g., von Willebrand factor). The CASMC and SVSMC were cultured on 100-mm or 150-mm culture dishes in Smooth Muscle Growth Media supplemented with 5% fetal calf sera, human recombinant epidermal growth factor, human recombinant fibroblast growth factor, insulin, gentamicin and amphotericin (SmGM-2; Cambrex) at 37° C. in a humidified atmosphere of 5% CO₂. Cells cultured at passage 6 and approximately 80% confluence were deprived of serum for 12 h, and then exposed to OxLDL (40 μg/ml) (BTI, Stoughton, Mass.), PDGF (10 ng/ml) (CalBioChem, San Diego, Calif.), or vehicle for 24 hrs.

The CAEC and SVEC were plated on 100-mm or 150-mm culture dishes in EGM-2MV media (Cambrex) containing 5% fetal calf sera, vascular endothelial growth factor (VEGF), basic fibroblast growth factor (bFGF), epidermal growth factor (EGF), hydrocortisone, ascorbic acid, gentamicin and amphotericin. The purity of endothelial cells was confirmed with Acetylated-LDL (Ac-LDL) up-take test (BTI). Cells at passage 6 and approximately 80% confluence were subjected to serum-free medium for 12 h and then exposed to oxidized-LDL (40μg/ml), TNFα (10 ng/ml), IL1β (10 ng/ml), PDGF (10 ng/ml) or vehicle for 24 hrs. Fully oxidized-LDL was purchased from BTI. TNFα, IL1β and PDGF were obtained from CalBioChem.

Cell Proliferation and Migration Assays. CAEC, SVEC, CASMC, and SVSMC were cultured overnight in serum-free media in 96-well plates at 37° C. in a humidified atmosphere of 5% CO₂. At about 80% confluence, cells were treated with OxLDL (40 μg/ml), PDGF (10 ng/ml), 10% FBS (fetal bovine serum), or vehicle for 24 hrs in the presence of bromodeoxyuridine (BrdU) for proliferation assays. The Cell Proliferation ELISA kit (Roche, Germany) was used according to the manufacturer's instructions. For the antibody immunoneutralization experiment, SVSMCs were first incubated with 25 μg/ml of polyclonal anti-IGF1 receptor (Upstate, Charlottesville, Va.) in serum-free medium for 24 h before adding indicated agents.

Cell migration assays were done using the InnoCyte™ Cell Migration Assay kit (Oncogene, San Diego, Calif.) in a 96-well plate. CASMC and SVSMC plated on the upper chambers migrated through the 8 μm pore-size of the membrane in response to vehicle, PDGF, OxLDL or FBS added to the lower chambers and incubated for 24 h at 37° C. in a humidified atmosphere of 5% CO₂. Migrated cells attached on the lower side of the membrane, were stained with a fluorescent dye, dislodged from the membrane, and quantified in a fluorescent reader. Each assay was performed in six replicate wells and performed independently three times.

### Results

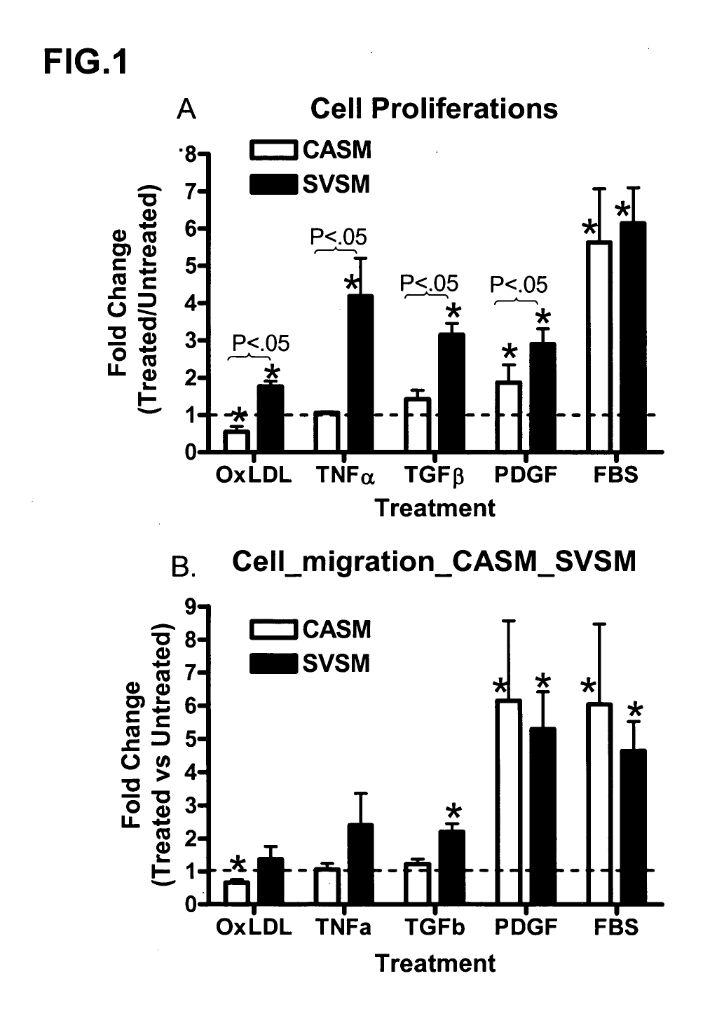

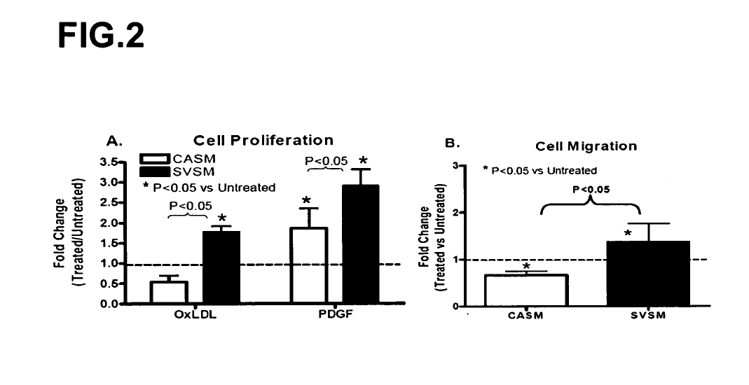

Cell proliferative and migratory responses to OxLDL, PDGF, and FBS were compared to establish cell viability and capacity to proliferate. FBS strongly induced increased proliferation and migration in both CASMC and SVSMC (FIGS. 1A & 1B), suggesting that both SMC subtypes used in these experiments are fully responsive. However, the CASMC and SVSMC proliferative and migratory responses to OxLDL and PDGF were different. OxLDL inhibited both proliferation and migration in CASMCs (FIGS. 2A & 2B) but significantly promoted proliferation in SVSMCs (FIG. 2A). PDGF also induced a greater proliferative response in SVSMC than CASMC (FIG. 1A), while the migratory response was similar (FIG. 1B). The stronger SVSM proliferative response to PDGF is consistent with a previous report of greater PDGF-induced growth in explant culture of saphenous vein than chest artery (Yang et al., 1998, Circulation 97: 181-187).

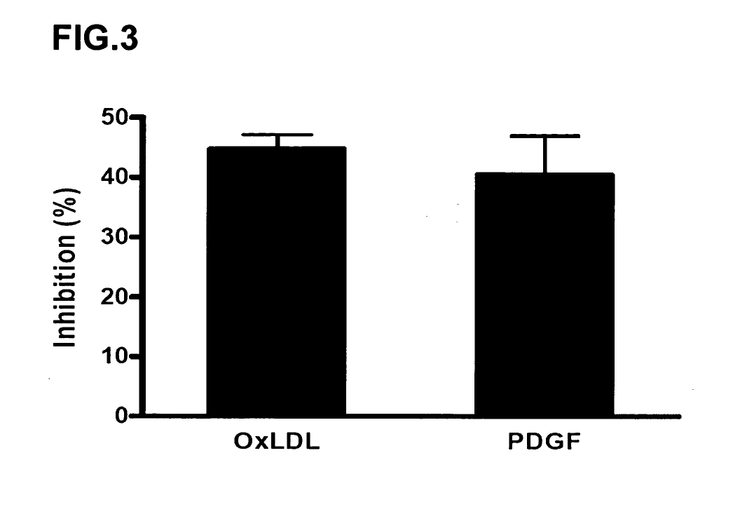

We further tested if IGF1 signaling plays a role in OxLDL-induced SVSMC proliferation. An antibody available from Upstate against the IGF1 receptor retarded SVSMC proliferation induced by OxLDL and PDGF by approximately 40-45% (FIG. 3). IGF1R antibody also reduced FBS-induced proliferation in SVSMC by 55% (data not shown).

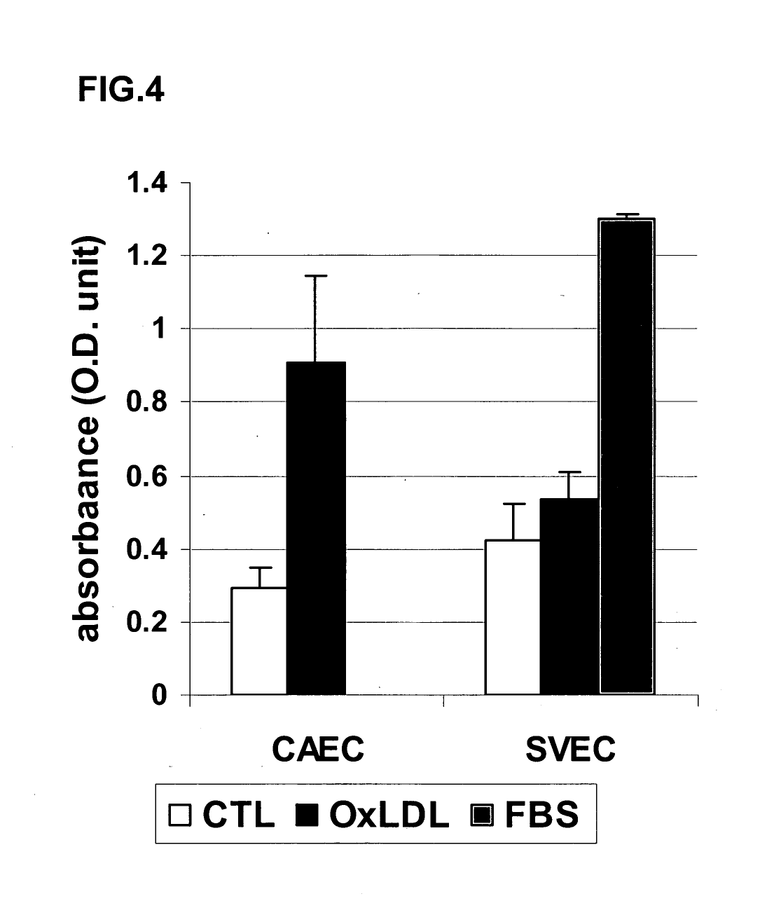

When the same number of CAEC or SVEC was plated on wells of the same 96-well plate, SVEC grew faster (about 1.7-fold) than CAEC in basal conditions (FIG. 4). In OxLDL-treated cells, OxLDL increased cell proliferation in CAEC by approximately 3-fold. In contrast, OxLDL induced no significant change in SVEC proliferation. However, FBS added after overnight serum-free culture to SVEC induced a 3-fold increase in proliferation. This verified that SVEC were viable and capable of undergoing cell division.

### Example 2

### Gene Expression of Smooth Muscle Cells

Arteries and veins are exposed to different hemodynamic environments in vivo. It is difficult to dissect the genetic contribution to different functional responses between venous and arterial cells in an in vivo model. To examine whether intrinsic differences between cells from different vascular beds could contribute to their different propensities to develop atherosclerosis, we compared gene expression profiles of endothelial cells and smooth muscle cells from saphenous veins and coronary arteries in response to various stimuli.

Gene expression profiling by high-density microarrays is a powerful tool for exploring complex interactive networks of genes and signaling pathways. However, identifying the appropriate biological context for large-scale changes in gene expression may be difficult, and the possible significance of the identification of a group of related genes as differentially regulated has not been adequately addressed with rigorous statistical methodology. Here, we have applied statistical analysis of gene ontology as previously published and used novel methods for analyzing pathway terms associated with regulated genes to better understand the full picture of gene expression differences observed in this study.

Primary cultures of vascular SMCs have been widely used in studies of molecular mechanisms underlying atherosclerosis. In this study, we used microarray gene expression profiling to identify the unique molecular signatures that distinguish human vascular smooth muscle cells (SMC) and endothelial cells (EC) subtypes from the saphenous vein (SV) and coronary artery (CA), and further characterized the molecular features that determine different proliferative and migratory responses to OxLDL, a key factor (Ross, 1999, N Engl J Med.) and PDGF, a potent mitogen and chemoattractant (Liu et al., 2004, J Surg Res.), and other stimuli.

### Materials and Methods

RNA isolation. Cells were harvested in Trizol reagent (Invitrogen) and immediately stored at −80° C. The aqueous layer after phenol-chloroform extraction was applied to an RNeasy® column (Qiagen, Valencia, Calif.) for further purification as per manufacturers instructions. The integrity of all samples was checked using the BioAnalyzer 2100 (Agilent, Palo Alto, Calif.).

Microarray Hybridization. Total RNA was isolated by known methods from the primary cell cultures at the end of the 24 hour incubation with OxLDL, PDGF, or vehicle. The isolated RNA and human universal RNA (Stratagene, La Jolla, Calif.), used as a control, were labeled with the Agilent Direct Fluorescent labeling kit (Palo Alto, Calif.) and hybridized to the Agilent human 1A oligo array representing 16,391 unique gene sequences (http://www.chem.agilent.com/). The gene sequences and identifiers for each sequence are available from Agilent. Total RNA from primary cell cultures and human universal RNA (Stratagene, La Jolla, Calif.) used as common reference for all samples, were labeled with the Agilent Direct Fluorescent labeling kit (Palo Alto, Calif.). The slides were scanned on an Agilent G2565AA Microarray Scanner System at 5% and 100% PMT settings to quantitate genes of both high and low signal intensities. Images were quantified using Agilent Feature Extraction Software (Version A. 7.1.2).

Data analysis. Data analysis was performed using Resolver® System (V4.0.1.0.1 0.RSPLIT) (Rosetta, Inc., Seattle, Wash.), by which the “raw ratios” (sample RNA versus common reference RNA for each condition and cell type) were technically separated into sample ratios and reference ratios for the further ratio analysis (first re-ratioed), and then the ratios were combined using an error-weighted approach wherein expression log ratios with less error contribute more to the combined result than those with greater error. Fold changes of gene expression greater than 1.5 and corresponding ANOVA P values ≦0.001 were considered significant and used in the following analyses.

Gene ontology (GO) analysis. Each list of differentially expressed genes was analyzed in the context of GO terms in order to identify groups of genes with similar functions or processes. GO annotation for genes represented on Agilent human 1A oligo arrays was obtained using Biomolecule Naming Service (Kincaid et al., BNS: An LDAP-based Biomolecule Naming Service. In: OiBC. Washington D.C.; 2002). BNS is a high-speed directory service developed at Agilent Laboratories, which can resolve between alias and official gene symbols, as well as between different gene identifier schemes, and links to publicly available databases. Of the total 16,400 genes on the array, 9828 genes annotated with GO terms were analyzed, with 8679 genes of cellular component, 7809 genes of biological process and 7562 genes of molecular function. In each specific comparison like treated vs non-treated cells, genes with fold change >1.5 and ANOVA P value <0.005 were identified, and applied GO and pathway analysis to this list of genes using similar methods as described by Doniger et al. (2003, Genome Biol. 4:R7).

Gene g was linked to a GO term t, if GO annotation for gene g contained term t or a child of term t. For each GO term t, we tested the hypothesis that term t is over- or under-represented in a given list of genes against the null hypothesis that the distribution of terms is random. This term to gene list association was measured using a hypergeometric distribution as the probability of observing k or more genes annotated by term t in the set of n genes, when there are K genes annotated by term t in the whole set of N genes. Namely, for each term t and list of genes l, the P value was calculated as follows:

$$p ( t , 1 ) = 1 - \frac{{\sum }_{y = 0}^{k} ( \begin{matrix}n \\ y\end{matrix} ) ( \begin{matrix}N - n \\ K - y\end{matrix} )}{( \begin{matrix}N \\ K\end{matrix} )} .$$

where:
N=the total number of unique genes represented in the microarray, n=the total number of unique genes represented in the set, l, K=the total number of entries in N mapping to a specific pathway, p, and k=the number of entries in n mapping to a specific pathway.

P values were Bonferroni corrected for multiple testing. Similar approaches for Gene Ontology analysis have been reported (Al-Shahrour et al., 2004, Bioinformatics. 20: 578-580; Zeeberg et al., 2003, Genome Biol. 4:R28). Terms with p<0.005 were considered significant.

Pathway analysis (PA). BNS was also used to link genes represented on the microarray to the list of 360 pathway terms associated with 1698 genes that was collected at Stanford from various sources such as Kyoto Encyclopedia of Genes and Genomes (KEGG), BioCarta, and Signaling PAthway Database (SPAD). The pathways were expressed as an Excel™ spreadsheet with each row representing a pathway name followed by the symbols for the pathway members. While the pathways consist of curated knowledge about various biological processes, they are not comprehensive, i.e., many genes or proteins measured in the microarray are not represented in terms of these curated pathways. A major source of protein interaction information that is being widely researched is scientific text.

To expand the list of gene symbols in each pathway in our database we used a prototype scientific literature association tool. Briefly, a large association network (an association represents a relation among genes or proteins extracted from a sentence of text) was automatically constructed using BioFerret and ALFA from over 250,000 PubMed abstracts. An extended pathway was constructed for each pathway in our database of 360 pathways as follows. For every gene symbol in a given pathway, all genes not in the pathway but associated with this gene via the literature association network were added to the extended pathway. The total number of 1357 genes was added to the individual pathways through the literature association network. The microarray data were then analyzed in terms of both the curated pathways and the extended pathways as follows. Given a subset of differentially expressed genes, l, and a list of pathways (each represented in terms of its genes), a statistical score was computed for each pathway, similar to the GO analysis. Since the pathway analysis was performed as part of an interactive user-guided process, we utilized the more computationally efficient Z scores instead of P values for the pathway analysis. Similar to Doniger et al., the score was calculated in terms of the statistical over- or under-occurrence of the genes from list l in each pathway.

$$Z ( p , l ) = \frac{( k - K \frac{n}{N} )}{\sqrt{K ( \frac{n}{N} ) ( 1 - \frac{n}{N} ) ( 1 - \frac{K - 1}{N - 1} )}} ,$$

where N=the total number of unique genes represented in the microarray, n=the total number of unique genes represented in the set, l, K=the total number of entries in N mapping to a specific pathway, p, and k=the number of entries in n mapping to a specific pathway.

The Z-score represents a surprise in finding “k” terms when we were expecting “nK/N” terms. A high positive value (signifying statistical over-abundance) or high negative value (signifying statistical under-abundance) of Z-score for a pathway implies a significant surprise level, and hence, interestingness of the pathway based on the experimental data. Based on the above-defined methods, each pathway was statistically scored for significance under different experimental conditions.

Based on the above-defined methods, each pathway was statistically scored for significance amongst the lists of differentially expressed genes from the different experimental conditions. A high Z score signified statistical over-abundance of genes in a specific pathway. A significant pathway was defined as Z scores of up regulated (top) or down-regulated (bottom) genes greater than 3.0 and with three or more genes associated the scientific literature. However, associations extracted automatically from scientific text can be inherently noisy. Almost every gene could be related to every other gene by a few sets of genes and associations. These genes are either regulators of very broadly defined processes or have been extensively reported in the literature. Nevertheless, this statistical approach reduced the noise effect since it discriminated against genes or terms that occur in a large number of pathways. We only reported the pathways with Z scores representing up-regulated or down-regulated genes greater than 3. To be consistent with GO analysis, we also converted the Z score to P-value. The number of gene counts for each significant PA term is listed (Table 1).

GO and PA terms with P value <0.005 were considered significant. To explore the impact of multiple testing correction, we applied Bonferroni correction to the P-values (Tables 1-2). However, Bonferroni correction assumes independence of the tests performed (Osier et al., 2004, BMC Bioinformatics. 5:124) and, given the complex dependency structure between terms in the GO hierarchy, Bonferroni adjusted P-values seem potentially overly conservative (Slonim, 2002, Nat Genet. 32 Suppl: 502-508). Indeed, we observed that many important biological terms with significant corresponding gene counts were excluded in the corrected data.

Application of GO terms in the analysis of microarray data has been published (Doniger et al., 2003), but statistical analysis of PA terms has not. GO and PA terms are associated with different sets of genes, with a total of 9828 genes associated with GO terms, and 3055 genes associated with PA terms. 2667 genes are associated with both GO and PA terms. Furthermore, the gene set associated with a PA term need not necessarily be associated with a GO term, and vice versa. For example, genes associated with PA term “Apoptosis_Homo_sapiens” were associated with multiple GO terms, like “induction of apoptosis”, “anti-apoptosis”, “regulation of apoptosis”, etc. Clearly, the PA and GO terms provide different kinds of biological information.

Results

A. Smooth Muscle Cells

OxLDL and PDGF differentially modulate growth-related gene expression between CASMC and SVSMC. Lists of differentially expressed genes from three independent primary cultures of CASMC and SVSMC cells both in the untreated state and in response to OxLDL and PDGF were generated by the criteria of 1.5-fold change in expression level with P values ≦0.001 as described above. OxLDL and PDGF elicited strong and dramatic differences in global gene expression responses and activated distinct signaling pathways between CASMC and SVSMC (Tables 3-7). There were 2262 genes (13.8%) altered by OxLDL in CASMC and 835 genes (5.1%) in SVSMC. Statistical analysis of Gene Ontology (GO) and Pathway (PA) terms revealed distinct pathways associated with these differentially expressed genes (Tables 8-9). In response to OxLDL, a set of 145 genes primarily related to stress, inflammatory and immune responses was altered in the same direction in both CASMC and SVSMC (Table 10). There were 76 genes with opposite changes between CASMC and SVSMC, including EDNRB, HMOX1, DCN, ABCA1, IGFB2 and IGFB4, IFI27, CTGF and SERPINE1 (Tables 11-12). Genes with consistent changes between in vitro cultured SMC and in vivo intact tissues from veins and arteries are displayed in Table 12.

In a separate study, we did gene expression profiling of intact tissues using custom oligo arrays containing approximately 20,500 genes mainly related to cardiovascular biology (unpublished data). These samples included 23 saphenous veins and 11 arteries (6 radial arteries, 5 internal mammary arteries) obtained from remnants of bypass surgery. We found dramatic differences in gene expression profiles between veins and arteries, with 635 genes differentially (p<0.005) expressed between the two types of tissues. Among these differentially expressed genes, the changes of 52 genes between intact venous and arterial tissues were consistent (changed in the same direction) with the changes seen with in vitro cultured CASM and SVSM. The overlapping genes were counted by matching the gene symbols between the Agilent catalog 1A arrays used for the analysis of the cells grown in culture containing probes representing approximately 16400 genes, and the custom cardiovascular arrays. It is possible that some genes were not matched because of annotation differences between the two arrays. Also, it is worth pointing out that intact vascular tissues, in contrast to pure SMCs in culture, are mixed populations of different cell types, with the most abundant cell type being SMC. Thus, the gene expression levels in intact tissues are the average of mixed cell populations within the tissue being tested.

There was a 36.6 and 21.8 fold difference in the expression of lumican and decorin, respectively, between venous and arterial smooth muscle cells.

TABLE 11
List of selected genes with opposite changes between CASMC and SVSMC in
response to OxLDL.

| | | CASM | | SVSM | |
|---|---|---|---|---|---|
| Symbol | GeneName | Ratio | P-value | Ratio | P-value |
| FDFT1 | Squalene synthase | −2.4 | 2.05E−19 | 1.6 | 3.72E−22 |
| HMOX1 | Heme oxygenase 1 | 6.5 | 9.47E−13 | −2.0 | 1.75E−15 |
| SERPINE1 | Plasminogen activator inhibitor 1 | −3.4 | 0.00E+00 | 1.5 | 1.81E−15 |
| INPP5D | SH2-containing inositol phosphatase | −3.2 | 6.57E−13 | 2.0 | 4.74E−12 |
| EGR1 | Early growth response 1 | 2.9 | 0.00E+00 | −1.9 | 8.76E−11 |
| DUSP3 | Dual specificity phosphatase 3 | −1.5 | 8.10E−04 | 1.5 | 9.33E−28 |
| DUSP1 | Dual specificity phosphatase 1 | 1.5 | 1.00E−04 | −1.5 | 1.55E−15 |
| S100A7 | S100 calcium-binding protein A7 | 2.9 | 2.80E−04 | −2.0 | 2.55E−15 |
| CCNA1 | Cyclin A1 | −1.8 | 5.70E−04 | 1.9 | 2.47E−11 |
| HRH1 | Histamine H1 receptor | −2.5 | 1.36E−07 | 1.5 | 4.26E−11 |
| CCKAR | Cholecystokinin A receptor | −1.8 | 6.10E−04 | 2.1 | 4.46E−11 |
| ADD3 | Adducin 3 (gamma) | 2.4 | 2.18E−26 | −1.7 | 1.01E−09 |
| IGFBP2 | Insulin like growth factor binding protein 2 | 4.2 | 7.84E−16 | −1.6 | 1.95E−08 |
| DCN | Decorin | 7.1 | 0.00E+00 | −2.2 | 4.17E−08 |
| ACTN1 | Alpha-actinin isoform 1 | −1.5 | 9.36E−11 | 1.6 | 4.51E−08 |
| EDNRB | Endothelin type B receptor | 15.9 | 5.71E−36 | −1.6 | 6.63E−08 |
| ACLY | ATP citrate lyase | −1.6 | 2.07E−17 | 1.8 | 1.33E−07 |
| ALDH1A1 | Aldehyde dehydrogenase 1 family member A1 | −2.4 | 3.82E−15 | 1.8 | 1.09E−06 |
| IFI27 | Interferon alpha-inducible protein 27 | −32.1 | 0.00E+00 | 2.1 | 1.36E−06 |
| SMURF2 | SMAD ubiquitination regulatory factor 2 | −1.9 | 3.00E−14 | 1.7 | 1.00E−05 |
| FN1 | Fibronectin | 2.8 | 1.26E−11 | −1.6 | 1.00E−05 |
| CTGF | Connective tissue growth factor | −14.1 | 0.00E+00 | 1.7 | 1.90E−04 |
| ABCA1 | ATP binding cassette subfamily A member 1 | 3.9 | 9.96E−27 | −1.9 | 7.50E−04 |

In particular, OxLDL differentially regulated several important growth-related genes and pathways, providing a possible molecular mechanism that may contribute to the phenotypic differences observed between CASMC and SVSMC.

1. Insulin Growth Factor Binding Proteins (IGFBPs). OxLDL upregulated IGFBP2, IGFBP3, IGFBP4, IGFBP5 and IGFBP6 in CASMC; but downregulated IGFBP2 and IGFBP4 in SVSMC (Tables 11 and 13). The regulation of IGFBP is consistent with the observed OxLDL-induced proliferation and migration between the two SMC subtypes (FIG. 2). There was no significant change in IGF1 and IGF1R expression Furthermore, PDGF-BB inhibited IGFBP2 but induced IGFBP3 in SVSMC but induced both IGFBP2 and IGFBP3 in CASMC (Table 13). Correspondingly, PDGF also caused greater cell proliferation in SVSMC than in CASMC (FIG. 2A).

OxLDL upregulated multiple isoforms of IGFBP genes in CASMC and down-regulated IGFBPs in SVSMC, which correlates well with the functional responses. Evidence suggests that IGFBPs may play an important role in the regulation of SMC growth and in atherogenesis by the inhibition of IGF activities and direct inhibitory effects (Scheidegger et al., 2000, J. Biol. Chem., 275:26864-26869; Delafontaine et al., 2004, Arterioscler. Thromb. Vasc. Biol., 24:435-444; Grant et al., 1996, Regul. Pept., 67:137-144; Zhang et al., 2002, Arterioscler. Thromb. Vasc. Biol., 22:2030-2036). For example, expression levels of IGFBP2 and other IGFBPs are increased in SMCs from atherosclerotic lesions (Grant et al., 1996; Zhang et al., 2002). Overexpression of IGFBP2 and IGFBP4 in SMCs in vitro inhibits proliferation and migration. Furthermore, transgenic overexpression of IGFBP2 and IGFBP4 in in vivo smooth muscle causes apoptosis and hypoplasia. The differential expression of IGFBPs between CASMC and SVSMC and the inhibition of SVSMC proliferation by an IGF1R antibody (Upstate, Charlottesville, Va.) support an important role for IGFBPs in determining proliferative responses between venous and arterial SMC. However, the fact that IGF1 receptor blockade only partially inhibited SMC proliferation suggests the direct effects of IGFBPs on SMC proliferation or/and other molecular mechanisms involved.

TABLE 13
IGFBP Genes Differentially Expressed in CASMC and SVSMC.

| Gene | Basal | CA | CA | SV | SV |
|---|---|---|---|---|---|
| Symbol | CA/SV | OxLDL | PDGF | OxLDL | PDGF |
| IGF1R | R | | | | |
| IGFBP2 | G | R | R | G | G |
| IGFBP3 | G | R | R | | R |
| IGFBP4 | G | R | | G | |
| IGFBP5 | | R | | | |
| IGFBP6 | G | R | | | |
| IGFBP7 | | R | | | |

Upregulated genes (R) over control, or genes having a higher level in untreated CASM;
Downregulated genes (G) over control, or genes having a higher level in untreated SVSM.
CA, CASM;
SV, SVSM.
Only significant genes with a P value ≦0.001 and fold change ≧1.5 are shown.
Blank cells indicate that the P value and fold change did not meet this requirement.

2. Cell cycle regulatory genes. OxLDL upregulated growth-inhibitory genes controlling cell cycling in CASMC, like cyclin-dependent kinase inhibitors CDKN1A, CDKN2C, CDKN2D, and cyclin-dependent kinase 3 (CDK3), while downregulating cyclin A (CCNA) and CCNA1 (Table 3). In contrast, OxLDL in SVSMC upregulated CDKN1A but downregulated cyclin A (Tables 4 and 11). These results suggest that transcriptional regulation of cell cycle gene expression may be involved in SMC proliferation. This is in agreement with the fact that IGF1R antibody only partially inhibited SVSMC proliferation (FIG. 3). OxLDL regulation of genes controlling cell cycle is consistent with the report that growth-inhibitory effects of OxLDL in arterial SMC is caused mainly by inhibition of nuclear translocation of cell cycle proteins, and not through apoptosis (Zettler et al., 2004, Arterioscler. Thromb. Vasc. Biol., 24: 727-732).

3. Phosphatidylinositol 3-kinase (PI3-K) and NF-κB pathways. Both PI3-K and NF-κB pathway associated genes were over-represented in the set of genes induced by OxLDL in SVSMC (Table 8), which agrees with our finding of OxLDL-induced cell proliferation and migration only in SVSMC (FIG. 2). The up-regulated genes associated with the PI3-K pathway in SVSMC included protein tyrosine phosphatase non-receptor type 14 (PTPN14), PTPN11, inositol polyphosphate-1-phosphatase (INPP1), and INPP5D, while down-regulated genes included dual specific phosphatase 1 (DUSP1) and protein-tyrosine phosphatase receptor type C (PTPRC or CD45), both negative regulators of cytokine receptor signaling. PI-3K and NF-κB pathways, associated with proliferation and migration, were overrepresented with upregulated genes only in SVSMC, while NF-κB was associated with down-regulated genes in CASMC. The outcome of NF-κB pathway activation by OxLDL can result in cell growth or inhibition, depending on cell type, OxLDL concentration and exposure time (Robbesyn et al., 2004, Free Radic. Res., 38:541-551). Taken together, these results suggest that different proliferative responses to OxLDL and PDGF between CASMC and SVSMC are mediated through regulation of IGF signaling (IGFBPs), genes controlling cell cycling, and PI3-K/NF-κB pathways.

OxLDL and PDGF modulate genes and molecular pathways involved in atherosclerosis in SMC. Several families of genes that may be associated with atherosclerosis were also differentially regulated by OxLDL and PDGF, including chemokines and cytokines, extracellular matrix (ECM) genes, genes related to prostaglandin pathways and genes specific for vascular cell types.

1. Chemokines and cytokines. OxLDL and PDGF differentially regulated several important cytokines and chemokines between CASMC and SVSMC (Tables 14-15). For example, chemokine CXCL12 was dramatically upregulated by OxLDL (44-fold) and PDGF in CASMC, whereas in SVSMC it was not changed by OxLDL but down-regulated by PDGF (Table 14). Both OxLDL and PDGF downregulated the expression of CXCL10 and CXCL11. Interestingly, the direction change of CXCL12 expression was opposite to its receptor CXCR4 (Table 14). OxLDL and PDGF also induced differential patterns of expression of cytokines and their regulatory proteins (Table 15).

TABLE 14
Chemokine Genes Differentially Expressed in CASMC and SVSMC.

| Gene | Basal | CA | CA | SV | SV |
|---|---|---|---|---|---|
| Symbol | CA/SV | OxLDL | PDGF | OxLDL | PDGF |
| CXCL12 | G | R | R | | G |
| CXCL10 | R | G | G | G | |
| CXCL11 | R | G | G | | |
| CXCL5 | R | | | | |
| CXCL1 | R | | | | |
| CXCL2 | R | | | | |
| CXCL3 | R | | | R | |
| CXCL6 | R | | | | |
| CXCL9 | R | | | R | |
| CXCR4 | R | G | | | R |
| CCL13 | G | R | | | |
| CCL17 | G | | | | |
| CCL22 | G | | | | |
| CCL24 | G | | | | |
| CCL2 | R | G | | | |
| CCL5 | | G | G | | |
| CCL14 | | G | | G | |
| CCL7 | | | R | | |
| CCL26 | | | R | | |
| CCL8 | | | | G | |
| CCL23 | | | | | G |

Upregulated genes (R) over control, or genes having a higher level in untreated CASM;
Downregulated genes (G) over control, or genes having a higher level in untreated SVSM.
CA, CASM;
SV, SVSM.
Only significant genes with a P value ≦0.001 and fold change ≧1.5 are shown.
Blank cells indicate that the P value and fold change did not meet this requirement.

2. Extracellular matrix (ECM) genes. Genes from several ECM gene families were differentially expressed between CASMC and SVSMC in response to OxLDL and PDGF (Table 16). Among MMP genes, MMP2 was the isoform most significantly upregulated in CASMC, while MMP10 was downregulated. Also, OxLDL and PDGF induced TIMP1 and TIMP2 expression in CASMC but not in SVSMC. OxLDL induced differential regulation of collagen (COL) and integrin genes, the major receptors for ECM-mediated cell adhesion, migration, proliferation and differentiation in CASMC but had effects on only a few of these genes in SVSMC (Table 16). Interestingly, decorin and fibronectin, two important ECM genes that negatively regulate cell growth, were significantly upregulated in CASMC but downregulated in SVSMC (Table 11), which is consistent with the SMC proliferative and migratory responses.

3. Endothelial and smooth muscle cell marker genes. In CASMC but not in SVSMC, OxLDL significantly (P<0.001) suppressed (by 1.7-44 fold) several endothelial cell (EC) marker genes including EDN1 (endothelin 1), VCAM1, PECAM1, CDH5, MMRN1, ESM1, LIPG, THBD and C1QR1. Interestingly, EDN receptor type B (EDNRB) was upregulated by OxLDL (Table 11). Furthermore, OxLDL in CASMC upregulated several smooth muscle genes including SM22-α, SM α-actin, vimentin, and ADD3, but not the cardiac muscle actin-α.

4. Prostaglandin (PTG) pathway genes. OxLDL significantly induced expression of prostaglandin-endoperoxide synthase 1 (COX1) in both CASMC and SVSMC (Table 10). However, induction of PTG receptors (PTGFR, PTGER3 and PTGD2R) was only seen in SVSMC (Tables 3 and 4), suggesting that prostaglandin signaling may play an important role in OxLDL-mediated effects in SVSMC.

SMC in vitro basal gene expression programs. CASMC and SVSMC in a basal state, deprived of serum for approximately 36 hrs, had clearly different gene expression patterns, with 1504 genes (9.2%) more highly expressed in CASMC, and 695 genes (10.3%) more highly expressed in SVSMC (Table 17).

Statistical analysis of pathway (PA) and gene ontology (GO) terms revealed that upregulated genes in pathways related to apoptosis, inflammation (CD40L, IL1R, CXR4, NF-κB) and lipid biosynthesis were significantly over-represented in CASMC (FIG. 3). On the other hand, upregulated genes related to cell growth, IGF binding, oxidoreductase activity, thrombin, extracellular matrix, fibrinolysis, and complement were significantly over-represented in SVSMC when compared to CASMC (FIG. 3). The relative expression levels of many of these genes seemed closely related to their responses to OxLDL and PDGF (Tables 13-16). Among these differentially expressed genes (Table 17), representatives of several particularly interesting gene families include the following:

1. IGFBPs. Four of the 5 IGFBPs expressed in human vascular SMC (IGFBP2, IGFBP3, IGFBP4 and IGFBP6) were significantly higher in SVSMC than in CASMC (Table 13). IGFBP3 had the highest differential expression (122-fold).

2. Chemokines and cytokines. Several proinflammatory cytokines (e.g. IL1α, IL6 and IL8) and α-Chemokine ligands (CXCL1, CXCL3, CXCL5, CXCL6 and CXCL8) had significantly higher expression levels in CASMC than in SVSMC (Table 14), with the exception of CXCL12 and its receptor CXCR4, which had markedly higher (5-100 fold) expression levels in CASMC than in SVSMC (Table 14). In contrast, four of the 5 β-chemokines (CCL17, CCL22, CCL24 and CCL28) were significantly expressed more highly in SVSMC than CASMC, with the exception being CCL2 (MCP-1), which was higher (by 28-fold) in CASMC than in SVSMC (Table 14).

3. Endothelial and SMC marker genes. Endothelial specific genes (Ho et al., 2003, Physiol. Genomics, 13:249-262) including EDN1, VCAM, PE-CAM1, MMRN1, ICAM1, and CDH5, had a relatively higher expression level (2-32 fold) in CASMC than in SVSMC (Table 18). THBD was an exception with 7-fold higher in SVSMC than in CASMC. Alternately, expression of several smooth muscle marker genes, including smoothelin, smooth muscle alpha actin 2 and vimentin, were higher in SVSMC than in CASMC (Table 18). Although the significance of endothelial marker gene expression in SMCs is unknown, the higher expression of some typical SMC markers in SVSMC may suggest that untreated venous smooth muscle cells exist in a more “differentiated” state. The suppression of endothelial genes and stimulation of SMC genes by OxLDL suggests phenotypic change of CASMC to a more differentiated state, which is consistent with OxLDL inhibiting CASMC proliferation and migration. The suppression of EDN1 may be due to OxLDL-induced upregulation (by 2.4-fold) of hepatocyte growth factor (Haug et al., 2000, Am. J. Physiol. Heart Circ. Physiol. 279: H2865-H2871). The dramatic upregulation of EDNRB in CASM by OxLDL supports the role of endothelin signaling in atherosclerosis (Haug et al., 2001, J Mol. Cell Cardiol. 33: 1701-1712).

4. Extracellular matrix (ECM) genes (Table 16). Notably, tissue inhibitor of metalloproteinase (TIMP) 1, TIMP2 and TIMP3 and matrix metalloproteinase (MMP) 2 were higher in SVSMC; whereas MMP10, MMP3, MMP20 and MMP26 were higher in CASMC. ECM genes play important roles in cell proliferation and migration. Higher levels of all three TIMPs in SVSMC could reduce the degradation of extracellular matrix, and thus inhibit proliferation and migration.

Venous SMC had stronger responses to OxLDL and PDGF than arterial SMC correlates with the fact that venous smooth muscle cells under stress, such as exposure to the arterial hemodynamic environment in bypass surgery, are prone to develop accelerated atherosclerosis, an important pathological process in vein graft disease (Motwani et al., 1998, Circulation 97: 916-931).

### Example 3

### Gene Expression of Endothelial Cells

The methods used to analyze the gene expression of endothelial cells, including data analysis for GO and PA, were conducted as described previously in Example 2.

### Results

Gene expression profiling. Five separate cultures of CAEC and SVEC from two different donors were treated with OxLDL, TNFα, IL1β, or PDGF. The cell cultures from the different donors were prepared to ensure that variations between donors and cell cultures did not significantly impact the data. All sample RNAs were labeled as red (cy5) channel, and universal human reference RNA as green (cy3) channel. Each treatment and cell type had at least 4 microarray hybridizations. Differentially expressed genes were selected on strict criteria of P value <0.005 and fold change >1.5 to reduce false positives. Reproducibility of microarray results as measured by the co-efficient of variation between arrays for signal intensities in the reference RNA channel was 10.5% for all probes and 6.8% for probes with signal intensities in the top 75%. Furthermore, expression level of five genes was selected to compare the results between microarrays and QuantiGene® assays in untreated and TNFα-treated cells. The results from the two methods were consistent with correlation of 0.98 (Table 19).

Baseline differences between gene expression profiles. Gene expression profiles of human coronary artery and human saphenous vein endothelial cell types were compared under basal conditions, in which cells were cultured with required survival factors without serum for 36 hours. The gene expression pattern was dramatically different between the two cell types. There were 1129 genes differentially (P<0.005) expressed between CAEC and SVEC (˜7%) (Table 20 and Table 31). When an arbitrarily chosen 1.5-fold cut-off was also applied, 285 genes were more highly expressed in SVEC, and 111 genes more highly expressed in CAEC.

Enrichment of atheroprotective genes in SVEC. The 111 genes over-expressed in CAEC were noticeably associated with inhibition of cell proliferation (e.g. IL6, IFNβ1, and necdin (NDN)), lipid metabolism (e.g. lipoprotein lipase (LPL), fatty acid binding protein 4 (FABP4), and collectin subfamily member 12 (COLEC12)).

The list of 285 genes over-expressed in SVEC was enriched for genes related to functions important for the development of atherosclerosis (Table 1 and FIG. 2), including:

(1) Regulation of cell growth. These genes included ESP8 and MIG-6, which are known to regulate EGFR signaling (Lanzetti et al., 2000, Nature 408: 374-377; Wick et al., 1995, Exp Cell Res. 219: 527-535.), and angiogenic factors such as ANGPT1 and VEGFC. In addition, many important transcription factor genes involved in cell proliferation and differentiation, like homeobox (HOX) D1, HOXD8, HOXA9, HOXA10 and HOXB7, were expressed at higher levels in SVEC (Table 20).

(2) Oxidoreductase activity and stress. Genes associated with the inhibition of reactive oxygen production were identified to be more highly expressed in SVEC, including RODH-4, COX6A2 and LOX (Table 21). In addition, CRYAB, a stress response gene, was 28-fold higher in SVEC compared to CAEC.

(3) Immune and anti-inflammatory responses. Several important genes directly or indirectly involved in the anti-inflammatory response and atherosclerosis were also more highly expressed in SVEC (Table 21). A factor encoded by one such gene, PAFAH2, hydrolyzes oxidized phospholipids and inactivates the strong pro-inflammatory mediator PAF (Peplow et al., 1999, Prostaglandins Leukot Essent Fatty Acids 61:65-82), and may reduce inflammatory injuries of SVEC. The gene for apolipoprotein E, a molecule important for clearance of circulating cholesterol and anti-inflammation, was >6 fold higher in SVEC than CAEC. Furthermore, five out of 9 pregnancy specific beta-glycoprotein (PSG) isoforms represented on our arrays (PSG1, 3, 6, 7 & 9), were more highly expressed in SVEC than CAEC. These factors have been recently linked to the anti-inflammatory responses (Blomberg et al., 1998, Proc Soc Exp Biol Med. 217: 212-218; Snyder et al., 2001, Am J Reprod Immunol. 45:205-216).

(4) Fibrinolysis. Thrombogenesis is an important process in coronary atherosclerosis as well as in deep vein thrombosis. A group of genes important for fibrinolysis and inhibition of thrombin formation, including PLAT, F2, TFPI and TFPI2, were more highly expressed in SVEC. The expression ratios were 1.8- to 6-fold (Table 21)

Statistical analysis of GO terms confirmed the over-representation of atheroprotective pathways such as “oxidoreductase activity” and “extracellular matrix” in SVEC (Table 22). PA term analysis also found that genes involved in “fibrinolysis” and “extrinsic prothrombin activation” pathways were over-represented in this list.

Markedly different gene expression responses to OxLDL in CAEC versus SVEC. We found OxLDL induced dramatically different gene expression responses between CAEC and SVEC. In CAEC, 122 genes were up-regulated and 175 genes down-regulated by OxLDL, while in SVEC, 267 genes were up-regulated and 550 genes down-regulated (P<0.005 and >1.5 fold). See Tables 23 and 24 for lists of all genes with P <0.005. Interestingly, only 36 genes induced by OxLDL were common between CAEC and SVEC. Bone morphogenetic protein 6 (BMP6) was among the genes up-regulated in both CAEC and SVEC, although the role of this potent growth and differentiation factor in vascular function is not known. Common down-regulated genes included several genes related to cholesterol/lipid synthesis like stearoyl-coenzyme A desaturase (SCD) and 3-hydroxy-3-methylglutaryl-Coenzyme A synthase (HMGCS1).

While little is known about the effect of OxLDL on SVEC, this stimulus had a potent effect on gene expression. There were 616 genes altered only in SVEC that were not significantly changed in CAEC, and 160 genes altered by OxLDL only in CAEC without concurrent change in SVEC. A gene without change was defined as up- or down-regulated with fold change <1.3 and P value >0.005. For example, OxLDL significantly inhibited TNFRSF5 (CD40) expression in SVEC but not in CAEC, which may reduce the inflammatory response to atherosclerotic stimuli in SVEC (Libby, 2003, Am J Cardiol. 91: 3A-6A). In addition, OxLDL down-regulated THBS4 in SVEC but up-regulated it in CAEC. Increased THBS4 activity has been associated with premature coronary artery disease and myocardial infarction (Topol et al., 2001, Circulation 104: 2641-2644).

OxLDL-induced expression of cellular proliferation genes in CAEC but not in SVEC. OxLDL induced sets of genes that regulate cellular proliferation only in CAEC. These genes included up-regulated genes known to promote cell growth, such as protein tyrosine kinase SYK (SYK), VGEF receptors neuropilin (NRP)1 and NRP2; and down-regulated genes that inhibit cell growth, like IGFBP3 (Bayes-Genis et al., 2000, Circ Res. 86: 125-130), fibroblast growth factor-inducible 14 (FN14) and IFN1β. Interestingly, we found OxLDL inhibited 8 out of 21 genes from the H1 histone family present on the arrays. Histone H1 is a linker of histone proteins involved in the condensation of chromatin. Down-regulation of H1 histone genes might decompress chromatin and facilitate cell proliferation. Paradoxically, we also found that OxLDL up-regulated pro-apoptotic genes in CAEC, such as CASP3, TNFSF10, THBS1 and THBS4, and down-regulated anti-apoptotic genes, like TNF receptor-associated factor 4 (TRAF4), inducible T-cell co-stimulator (ICOS) and PIM1.

Identification of OxLDL-activated pathways that distinguish CAEC and SVEC. By analyzing GO and PA terms, we found some pathways significantly over-represented by OxLDL regulated genes in CAEC but not in SVEC (Tables 25-26). For example, cell adhesion pathways were not significantly up-regulated in SVEC but were in CAEC, consistent with the previous report that OxLDL does not induce adhesion molecule expression in SVEC, but does in arterial EC (Amberger et al., 1997, Cell Stress Chaperones, 2:94-103). Furthermore, terms for the regulation of focal adhesion and inflammatory response in SVEC were associated with OxLDL-induced down-regulated genes (Table 25). Similarly, genes down-regulated by OxLDL in SVEC were significantly associated with pathways known to be important in atherosclerosis, such as “induction of apoptosis,” the “NF-KB pathway” and “regulation of cell cycle” (Table 26). These results further support that some genes related to protective pathways are activated in SVEC.

In arterial cells, OxLDL activated pathways related to Alzheimer's disease (Table 26). These genes included SYK, pyruvate dehydrogenase kinase 4 (PDK4), microtubule-associated protein 2 (MAP2), calmodulin 3 (calm3), CASP3, and aldehyde dehydrogenase 6 (ALDH1α3). The potential link between Alzheimer's disease and atherosclerosis has been recently suggested in a mouse model (Li et al., 2003, Am J Pathol. 163: 2155-2164).

Strong gene expression responses to TNFα and IL1β in both CAEC and SVEC. TNFα and IL1β are classical pro-inflammatory cytokines. The robust and similar gene expression response of CAEC and SVEC to TNFα and IL1β was in direct contrast to their significantly different response to OxLDL. Overall, CAEC had 1108 and 1130 genes that showed significant (P<0.005 and >1.5 fold) differential expression responses to TNFα and IL1β, respectively, while SVEC had 968 and 829 genes that responded (Tables 27-30). Of these, a common set of 210 genes was up-regulated and 42 genes down-regulated by both TNFα and IL1β in CAEC and SVEC. Reassuringly, many genes known to be induced by TNFα and IL1β were near the top of the lists when sorted by fold change, including IL8, SERPINE1 (PAI1), E selectin (SELE), superoxide dismutase 2 (SOD2), TNFα inducible protein 6, ICAM1, VCAM1, IL6, and chemokine ligand 3 (CXCL3). Prominent TNFα and IL1β down-regulated genes included matrix Gla protein (MGP), thrombomodulin (THBD), and CXC chemokine receptor 4 (CXCR4).

GO term analysis yielded a similar pattern of significant terms for IL1β and TNFα in both CAEC and SVEC, including pathways known to be associated with TNFα and IL1β. These terms included “inflammatory response,” “apoptosis,” “cell proliferation,” “immune response” and “cytokine/chemokine” (Table 25). Similarly, PA term analysis found many pathways known to be associated with TNFα were significantly over-represented, including “apoptosis”, “cytokine and inflammatory response,” “NF-κb signaling,” “toll like receptor,” “IL6, IL1 and IFN signaling” pathways (Table 26).

Further comparison of TNFα-induced gene expression profiles between CAEC and SVEC revealed a set of differentially responsive genes between the two cell types. There was a set of 810 genes in common regulated by TNFα in CAEC and SVEC while 283 and 162 genes were altered by TNFα only in CAEC or SVEC, respectively. These differences may be important. For example, in CAEC, TNFα up-regulated apoptosis genes, e.g. CASP1, and down-regulated anti-inflammatory genes, e.g. PAFAH2, but this effect was not seen in SVEC. GO terms related to anti-apoptosis, an anti- process, were significantly over represented in genes regulated by TNFα and IL1β only in SVEC. Several pathways that mediate anti-processes like IL4 and inhibition of cellular proliferation were significantly represented only in TNFα regulated genes in SVEC. The finding that some anti-pathways are activated by TNFα and IL1β in SVEC but not in CAEC suggests that relatively subtle differences in the ability of venous versus arterial cells to respond to these cytokines.

### Example 4

### Chemokines CXCL10, CXCL1 and CXCL12 New Diagnostic Markers for Atherosclerosis

Chemokines are potent activators and chemoattractants of leukocytes and are implicated in many disease processes. However, it has not been reported that specific chemokines CXCL10, CXCL11 and CXCL12 are expressed in the vascular cells, nor has it been shown that they are regulated by oxidized LDL (OxLDL), a key factor. We found that OxLDL, and cytokines TNFα and IL1β as well as growth factors PDGF and TGFβ markedly induced the expression of CXCL12, but suppressed the expression of CXCL10 and CXCL11 in in vitro primary culture of coronary artery smooth muscle cells. Chemokines are secretory proteins. These specific chemokines directly involved in vascular atherosclerotic process could be important new diagnostic markers for the cardiovascular disease.

CXCL10, CXCL11, and CXCL12 (also called stromal cell-derived factors 1, SDF1) belong to a subfamily of α chemokines. CXCL11 is a ligand for CXCR3 and an angiostatic ELR chemokines. CXCL11 and CXCR3 may have an important role in the metastasis of lymphoma and melanoma as well as pulmonary fibrinosis (Burdick et al., 2004, Am. J Respir. Crit. Care Med.; Kawada et al., 2004, Cancer Res. 64: 4010-4017). CXCR3 is also the receptor for CXCL9 and CXCL10. CXCL10 (alternative names C7, IFI10, INP10, IP-10, crg-2, mob-1, SCYB10, gIP-10) through binding to CXCR3 induces pleiotropic effects, including stimulation of monocytes, natural killer and T-cell migration, and modulation of adhesion molecule expression.

CXCL12 is a ligand for chemokine receptor CXCR4. CXCR4 is known to be involved in fusion of human immunodeficiency virus-1 (HIV-1) with CD4+ lymphocytes. The functional role of CXCL12/CXCR4 has been intensively studied, including:

CXCL12 is a highly efficacious lymphocyte chemoattractant, and a potent inhibitor in vitro of infection by lymphocyte-tropic HIV-1 strains. CXCL12 is also a substrate for the matrix metalloproteinase-2 (MMP2), which cleaves a CXCL12 N-terminal tetrapeptide. The cleaved protein of CXCL12 showed loss of CXCR4 binding affinity and was unable to block CXCR4-dependent HIV infection in lymphocytes. The association between a common polymorphism of CXCL12 (SDF1-3′ A) and HIV invection has been intensively studied (Tiensiwakul, 2002, Asian Pac. J. Allergy Immunol, 20:61-65; Soriano et al., 2002, J. Infect., Dis., 186:922-931; Roman et al., 2002, HIV Clin. Trials, 3:195-201; Ryabov et al., 2004, Genet. Test, 8:73-76; Singh et al., 2003, J Infect. Dis., 188:1461-1472).

Several studies have demonstrated that CXCL12 mobilizes cell migration and modulates cell homing and survival, including haematopoietic stem cells, neuronal cells, and germ cells (Hattori et al., 2003, Leuk Lymphoma, 44: 575-582; Molyneaux et al., 2003, Develop., 130: 4279-4286; Cyster, 2003, Immunol. Rev., 194:48-60; David et al., 2002, Proc. Natl. Acad. Sci. USA, 99:16297-16302; Barbero et al., 2002, Ann. NY Acad. Sci., 973:60-69).

CXCL12 and CXCR4 have been reported to highly express in lymphoma and breast cancer cells and may be associated with tumor cell metastasis (Muller et al, 2001, Nature 410: 50-56; de Oliveira Cavassin et al., 2004, Blood Cells Mol. Dis., 33:90-93; Sei et al., 2001, Cancer Res., 61:5028-5037; Rabkin et al., 1999, Blood, 93:1838-1842).

Recently, the implication of CXCL12 in diabetes has been reported, especially the association of genetic polymorphisms of CXCL12 (SDF1-3′ A) with early onset of type I diabetes (Ide et al., 2003, Hum Immunol., 64:973-978; Dubois-Laforgue et al., 2001, Diabetes, 50:1211-1213).

In summary, chemokines CXCL10, CXCL11 and CXCL12 have been implicated in many disease states. Surprisingly, the role of these chemokines in cardiovascular cells, especially in the setting of atherosclerosis, has not been studied.

Gene Expression Profiling. We performed gene expression profiling using Agilent 22K oligonucleotide arrays of in vitro cultured smooth muscle cells (SMC) and endothelial cells (EC) isolated from normal coronary artery and saphenous vein both in basal state (cell cultured under serum-free medium for 36 hrs) and in responses to athero-related stimuli, Oxidized LDL, TNFα, IL1β, PDGF, TGFβ and TNFα.

### Results

Responses of chemokines to stimuli in the SMCs from coronary artery (CASM) and saphenous vein (SVSM) produced differential expression.

In CASM, the OxLDL dramatically induced (by 44 fold, P-value=1E-16) the expression of CXCL12 (on the top of differentially expressed gene list), but significantly suppressed the expression of CXCR4 by approximately 2 fold, the receptor for CXCL12. Furthermore, IL1β, TNFα, PDGF and TGFβ also up-regulated CXCL12 in CASM. In contrast, OxLDL, IL1β, TNFα, PDGF and TGFβ inhibited the expression of CXCL11 and CXCL10. In coronary artery endothelial cells, OxLDL also up-regulated CXCL12 (data not shown).

Notably, the responses of chemokines to stimulations between CASM and SVM were quite different, suggesting inherent differences between the two SMCs. For example, PDGF suppressed CXCL12 expression in SVSM, which was opposite to the change in CASM. Similarly, effects of TNFα on the expression of CXCL10 and CXCL11 were opposite between CASM and SVSM, with dramatically up-regulated in SVSM but down-regulated in CASM.

### Example 5

### qRT-PCR

Quantitative real-time RT-PCR. Total RNA (5 μg) from the same preparations used for the microarray experiments in Example 1 was reverse transcribed to cDNA using MMLV reverse transcriptase. The cDNA was amplified in triplicate using an ABI PRISM® 7900HT thermocycler with Taqman® (Applied Biosystems, Foster City, Calif.) primers and probes (dual-labeled probes to fluoresce upon 5′→3→ exonuclease activity). Using a hot start, the samples were kept on ice until the ABI PRISM® 7900HT thermocycler reached its initial cycling temperature of 50° C. and then the PCR tubes were placed in the thermal cycler. The samples were incubated at 50° C. for 3000 sec followed by Taq DNA Polymerase activation at 95° C. for 900 sec. These incubations in the thermal cycler were followed by 45 cycles of 95° C. for 10 sec, 53° C. for 25 sec, and 62° C. for 70 sec. Fluorescence was read at the end of each elongation step. Gene expression levels were normalized to corresponding 18S internal controls.

### Results

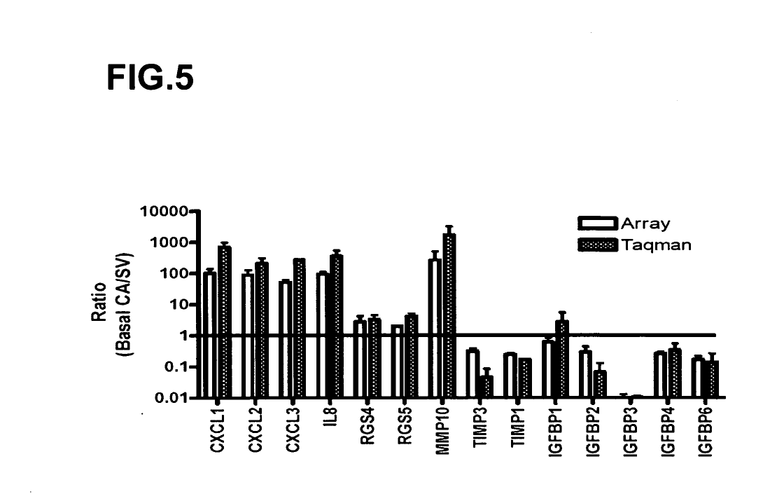

Fourteen genes were subject to Taqman Real-Time PCR for the same RNA samples used in our microarray hybridization. The relative ratios from Taqman analysis correlated well to array hybridization results, with a correlation coefficient of 0.88 (FIG. 5). Furthermore, the differential expression levels between CASMC and SVSMC of certain genes were consistent with relative expression levels in in vivo tissues between human arteries (radial and internal mammary arteries) and saphenous veins. For example, genes related to ECM, like decorin, lumican, TIMP3, COL6A3, and CD44, were higher in intact veins than arteries, while several genes related to cellular signaling, like RGS5, RAC2, DAF, G3BP and stathmin1, were higher in arteries.

What is claimed:

1. A method for diagnosing cardiovascular disease or susceptibility to cardiovascular disease comprising:
detecting a CXCL12 polypeptide in a biological sample, wherein an increase in CXCL12 polypeptide as compared to control sample is indicative of cardiovascular disease.

2. The method of claim 1, wherein the CXCL12 polypeptide is detected using an antibody.

3. The method of claim 1, wherein the cardiovascular condition is selected from the group consisting of atherosclerosis, stroke, ischemic heart disease, hypertension, cardiac hypertrophy, angina, and coronary heart disease.

4. The method of claim 1, wherein the CXCL12 polypeptide is detected by an activity assay.

5. The method of claim 4, wherein the activity assay measures chemotactic activity for lymphocytes of CXCL12.

6. The method of claim 4, wherein the activity assay measures the cleavage of CXCL12 by matrix metalloproteinase-2.

7. The method of claim 1, wherein the biological sample is blood or serum.

8. The method of claim 1, wherein the biological sample comprises aortic smooth muscle cells.

9. The method of claim 1, wherein the biological sample comprises aortic endothelial cells.

10. The method of claim 1, further comprising detecting a CXCL11 or CXCL10 polypeptide or both, wherein a decrease in the CXCL11 or CXCL10 polypeptide or both as compared to a control is indicative of cardiovascular disease.

11. The method of claim 10, wherein the detecting CXCL11 or CXCL10 polypeptide, or both, comprising detecting the polypeptide with a specific antibody that binds to CXCL11 or CXCL10, respectively.

12. The method of claim 10, wherein the biological sample is blood or serum.

13. The method of claim 10, wherein the biological sample comprises aortic smooth muscle cells.

14. The method of claim 10, wherein the biological sample comprises arterial endothelial cells.

15. A method for screening for an antagonist of CXCL12 comprising:
a) contacting a CXCL12 polypeptide with an agent; and
b) determining whether the agent inhibits binding of CXCL12 to its receptor CXCR4 or inhibits an activity of the CXCL12.

16. The method of claim 15, wherein the agent is an antibody.

17. The method of claim 15, wherein the agent is an aptamer.

18. The method of claim 15, wherein the activity of CXCL12 is chemoattraction of lymphocytes.

19. The method of claim 15, wherein the activity of CXCL12 is cleavage of CXCL12 by matrix metalloproteinase-2.

20. A method for screening for an antagonist of CXCL12 comprising contacting polynucleotide encoding a CXCL12 polypeptide, with a candidate agent, and determining whether the candidate agent inhibits expression of the polypeptide.

21. The method of claim 20, wherein the agent is an antisense molecule, or siRNA.

22. The method of claim 20, wherein the agent is a peptide nucleic acid.

23. The method of claim 20, wherein expression of the polynucleotide is detected with a probe.

24. The method of claim 20, wherein the expression of the polynucleotide is detected with a primer and PCR.

25. A method for diagnosing cardiovascular disease or susceptibility to cardiovascular disease comprising:
detecting expression of a polynucleotide encoding a CXCL12 polypeptide in a biological sample, wherein an increase in expression of a CXCL12 polypeptide as compared to control sample is indicative of cardiovascular disease.

26. The method of claim 25, wherein the expression of a CXCL12 polypeptide is detected using an antibody.

27. The method of claim 25, wherein the cardiovascular condition is selected from the group consisting of atherosclerosis, stroke, ischemic heart disease, hypertension, cardiac hypertrophy, angina, and coronary heart disease.

28. The method of claim 25, wherein the expression of a CXCL12 polypeptide is detected by an activity assay.

29. The method of claim 28, wherein the activity assay measures chemotactic activity for lymphocytes of CXCL12.

30. The method of claim 28, wherein the activity assay measures the cleavage of CXCL12 by matrix metalloproteinase-2.

31. The method of claim 25, wherein the biological sample is blood or serum.

32. The method of claim 25, wherein expression of the polynucleotide is detected with a probe.

33. The method of claim 25, wherein the expression of the polynucleotide is detected with a primer and PCR

34. The method of claim 25, further comprising detecting expression of a polynucleotide encoding a CXCL11 or CXCL10 polypeptide or both, wherein a decrease in the expression of a CXCL11 or CXCL10 polypeptide or both as compared to a control is indicative of cardiovascular disease.

35. The method of claim 34, wherein the detecting expression of a polynucleotide encoding a CXCL11 or CXCL10 polypeptide, or both, comprises detecting the polypeptide with a specific antibody that binds to CXCL11 or CXCL10, respectively.

36. The method of claim 34, wherein the biological sample is blood or serum.

37. The method of claim 34, wherein expression of the polynucleotide encoding a CXCL11 or CXCL10 polypeptide or both is detected with a probe.

38. The method of claim 34, wherein the expression of the polynucleotide encoding a CXCL11 or CXCL10 polypeptide or both is detected with a primer and PCR
